SEQUENCE LISTING (61 sequences)

SEQ ID NO: 1 - protein, 24 aa, Homo sapiens
QLYSALANKCCHVGCTKRSLARFC

SEQ ID NO: 2 - protein, 29 aa, Homo sapiens
DSWMEEVIKLCGRELVRAQIAICGMSTWS

SEQ ID NO: 3 - protein, 25 aa, Mus musculus
ESGGLMSQQCCHVGCSRRSIAKLYC

SEQ ID NO: 4 - protein, 35 aa, Mus musculus
RVSEEWMDGFIRMCGREYARELIKICGASVGRLAL

SEQ ID NO: 5 - protein, 24 aa, Artificial Sequence
  Description of Artificial Sequence Synthetic peptide
DLYSALANKCCHVGCTKRSLARFC

SEQ ID NO: 6 - protein, 24 aa, Artificial Sequence
  Description of Artificial Sequence Synthetic peptide
EVIKLCGRELVRAQIAICGMSTWS

SEQ ID NO: 7 - protein, 29 aa, Artificial Sequence
  Description of Artificial Sequence Synthetic peptide
DSWKEEVIKLCGRELVRAQIAICGMSTWS

SEQ ID NO: 8 - protein, 29 aa, Artificial Sequence
  Description of Artificial Sequence Synthetic peptide
DSWMEEVIKLCGRELVRAQIAICGKSTWS

SEQ ID NO: 9 - protein, 29 aa, Artificial Sequence
  Description of Artificial Sequence Synthetic peptide
DSWMEEVIKLCGRELVRAQIAICGMSTAS

SEQ ID NO: 10 - protein, 29 aa, Artificial Sequence
  Description of Artificial Sequence Synthetic peptide
DSWKEEVIKLCGRELVRAQIAICGKSTAS

SEQ ID NO: 11 - protein, 29 aa, Artificial Sequence
  Description of Artificial Sequence Synthetic peptide
DSWKEEVIKLCGDELVDAQIAICGKSTAS

SEQ ID NO: 12 - protein, 29 aa, Artificial Sequence
  Description of Artificial Sequence Synthetic peptide
DSWKEEVIKLCGDELVDAQDAICGKSTAS

SEQ ID NO: 13 - protein, 29 aa, Artificial Sequence
  Description of Artificial Sequence Synthetic peptide
DSWKEEVIKLCGAELVAAQAAICGKSTAS

SEQ ID NO: 14 - protein, 12 aa, Artificial Sequence
  Description of Artificial Sequence Synthetic peptide
DAASSHSHSSAR

SEQ ID NO: 15 - protein, 12 aa, Artificial Sequence
  Description of Artificial Sequence Synthetic peptide
DAASSHSHSSAA

SEQ ID NO: 16 - protein, 12 aa, Artificial Sequence
  Description of Artificial Sequence Synthetic peptide
DAAGANANAGAR

SEQ ID NO: 17 - protein, 12 aa, Artificial Sequence
  Description of Artificial Sequence Synthetic peptide
GGSGGSGGSGGS

SEQ ID NO: 18 - protein, 6 aa, Artificial Sequence
  Description of Artificial Sequence Synthetic 6xHis tag
HHHHHH

SEQ ID NO: 19 - protein, 8 aa, Artificial Sequence
  Description of Artificial Sequence Synthetic 8xHis tag
HHHHHHHH

SEQ ID NO: 20 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 590 aa, Mus musculus
RGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 23 - protein, 8 aa, Artificial Sequence
  Description of Artificial Sequence Synthetic peptide
LEVLFQGP

SEQ ID NO: 24 - protein, 14 aa, Artificial Sequence
  Description of Artificial Sequence Synthetic peptide
EDQVDPRLIDGKGS

SEQ ID NO: 25 - protein, 71 aa, Artificial Sequence
  Description of Artificial Sequence Synthetic polypeptide
HHHHHHDSWMEEVIKLCGRELVRAQIAICGMSTWSDAASSHSHSSARQLYSALANKCCHVGCTKRSLARFC

SEQ ID NO: 26 - protein, 71 aa, Artificial Sequence
  Description of Artificial Sequence Synthetic polypeptide
HHHHHHDSWMEEVIKLCGRELVRAQIAICGMSTWSDAASSHSHSSAADLYSALANKCCHVGCTKRSLARFC

SEQ ID NO: 27 - protein, 82 aa, Artificial Sequence
  Description of Artificial Sequence Synthetic polypeptide
HHHHHHGSLEVLFQGPGDSWMEEVIKLCGRELVRAQIAICGMSTWSDAASSHSHSSARQLYSALANKCCHVGCTKRSLARFC

SEQ ID NO: 28 - protein, 85 aa, Artificial Sequence
  Description of Artificial Sequence Synthetic polypeptide
EDQVDPRLIDGKGSHHHHHHDSWMEEVIKLCGRELVRAQIAICGMSTWSDAASSHSHSSARQLYSALANKCCHVGCTKRSLARFC

SEQ ID NO: 29 - protein, 67 aa, Artificial Sequence
  Description of Artificial Sequence Synthetic polypeptide
HHHHHHEEVIKLCGRELVRAQIAICGMSTWSDAASSHSHSSARQLYSALANKCCHVGCTKRSLARFC

SEQ ID NO: 30 - protein, 76 aa, Artificial Sequence
  Description of Artificial Sequence Synthetic polypeptide
HHHHHHLEVLFQGPGEEVIKLCGRELVRAQIAICGMSTWSDAASSHSHSSARQLYSALANKCCHVGCTKRSLARFC

SEQ ID NO: 31 - protein, 298 aa, Artificial Sequence
  Description of Artificial Sequence Synthetic polypeptide
DSWMEEVIKLCGRELVRAQIAICGMSTWSDAASSHSHSSARQLYSALANKCCHVGCTKRSLARFCGSGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 295 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGMSTWSDAASSHSHSSARQLYSALANKCCHVGCTKRSLARFC

SEQ ID NO: 33 - protein, 304 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSDSWMEEVIKLCGRELVRAQIAICGMSTWSDAASSHSHSSARQLYSALANKCCHVGCTKRSLARFC

SEQ ID NO: 34 - protein, 295 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGMSTWSDAASSHSHSSARQLYSALANKCCHVGCTKRSLARFC

SEQ ID NO: 35 - protein, 295 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAADSWMEEVIKLCGRELVRAQIAICGMSTWSDAASSHSHSSARQLYSALANKCCHVGCTKRSLARFC

SEQ ID NO: 36 - protein, 295 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKPPPDSWMEEVIKLCGRELVRAQIAICGMSTWSDAASSHSHSSARQLYSALANKCCHVGCTKRSLARFC

SEQ ID NO: 37 - protein, 295 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRAQIAICGMSTWSDAASSHSHSSARQLYSALANKCCHVGCTKRSLARFC

SEQ ID NO: 38 - protein, 295 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGKSTWSDAASSHSHSSARQLYSALANKCCHVGCTKRSLARFC

SEQ ID NO: 39 - protein, 295 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGMSTASDAASSHSHSSARQLYSALANKCCHVGCTKRSLARFC

SEQ ID NO: 40 - protein, 295 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGMSTWSDAAGANANAGARQLYSALANKCCHVGCTKRSLARFC

SEQ ID NO: 41 - protein, 295 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRAQIAICGKSTASDAAGANANAGARQLYSALANKCCHVGCTKRSLARFC

SEQ ID NO: 42 - protein, 292 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDSWKEEVIKLCGRELVRAQIAICGKSTASDAAGANANAGARQLYSALANKCCHVGCTKRSLARFC

SEQ ID NO: 43 - protein, 295 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGDELVDAQIAICGKSTASDAAGANANAGARQLYSALANKCCHVGCTKRSLARFC

SEQ ID NO: 44 - protein, 295 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGDELVDAQDAICGKSTASDAAGANANAGARQLYSALANKCCHVGCTKRSLARFC

SEQ ID NO: 45 - protein, 295 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGAELVAAQAAICGKSTASDAAGANANAGARQLYSALANKCCHVGCTKRSLARFC

SEQ ID NO: 46 - protein, 666 aa, Artificial Sequence
  Description of Artificial Sequence Synthetic polypeptide
HHHHHHHHRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGSDSWKEEVIKLCGRELVRAQIAICGKSTASDAAGANANAGARQLYSALANKCCHVGCTKRSLARFC

SEQ ID NO: 47 - protein, 65 aa, Artificial Sequence
  Description of Artificial Sequence Synthetic polypeptide
DSWMEEVIKLCGRELVRAQIAICGMSTWSDAASSHSHSSARQLYSALANKCCHVGCTKRSLARFC

SEQ ID NO: 48 - protein, 65 aa, Artificial Sequence
  Description of Artificial Sequence Synthetic polypeptide
DSWMEEVIKLCGRELVRAQIAICGMSTWSDAASSHSHSSAADLYSALANKCCHVGCTKRSLARFC

SEQ ID NO: 49 - protein, 61 aa, Artificial Sequence
  Description of Artificial Sequence Synthetic polypeptide
EEVIKLCGRELVRAQIAICGMSTWSDAASSHSHSSARQLYSALANKCCHVGCTKRSLARFC

SEQ ID NO: 50 - protein, 65 aa, Artificial Sequence
  Description of Artificial Sequence Synthetic polypeptide
DSWKEEVIKLCGRELVRAQIAICGMSTWSDAASSHSHSSARQLYSALANKCCHVGCTKRSLARFC

SEQ ID NO: 51 - protein, 65 aa, Artificial Sequence
  Description of Artificial Sequence Synthetic polypeptide
DSWMEEVIKLCGRELVRAQIAICGKSTWSDAASSHSHSSARQLYSALANKCCHVGCTKRSLARFC

SEQ ID NO: 52 - protein, 65 aa, Artificial Sequence
  Description of Artificial Sequence Synthetic polypeptide
DSWMEEVIKLCGRELVRAQIAICGMSTASDAASSHSHSSARQLYSALANKCCHVGCTKRSLARFC

SEQ ID NO: 53 - protein, 65 aa, Artificial Sequence
  Description of Artificial Sequence Synthetic polypeptide
DSWKEEVIKLCGRELVRAQIAICGKSTASDAASSHSHSSARQLYSALANKCCHVGCTKRSLARFC

SEQ ID NO: 54 - protein, 65 aa, Artificial Sequence
  Description of Artificial Sequence Synthetic polypeptide
DSWMEEVIKLCGRELVRAQIAICGMSTWSDAAGANANAGARQLYSALANKCCHVGCTKRSLARFC

SEQ ID NO: 55 - protein, 65 aa, Artificial Sequence
  Description of Artificial Sequence Synthetic polypeptide
DSWKEEVIKLCGRELVRAQIAICGKSTASDAAGANANAGARQLYSALANKCCHVGCTKRSLARFC

SEQ ID NO: 56 - protein, 6 aa, Artificial Sequence
  Description of Artificial Sequence Synthetic peptide
LVPRGS

SEQ ID NO: 57 - protein, 6 aa, Artificial Sequence
  Description of Artificial Sequence Synthetic peptide
GSGGSG

SEQ ID NO: 58 - protein, 11 aa, Artificial Sequence
  Description of Artificial Sequence Synthetic peptide
GSLEVLFQGPG

SEQ ID NO: 59 - protein, 5 aa, Homo sapiens
DSWME

SEQ ID NO: 60 - protein, 295 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRSQIAICGKSTASDAAGANANAGARQLYSALANKCCHVGCTKRSLARFC

SEQ ID NO: 61 - protein, 295 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRGQIAICGKSTASDAAGANANAGARQLYSALANKCCHVGCTKRSLARFC

(19) United States
(12) Patent Application Publication
(10) Pub. No.: US 2023/0174610 A1
(43) Pub. Date: Jun. 8, 2023

(54) ENGINEERED RELAXINS AND METHODS OF USE THEREOF

(71) Applicant: President and Fellows of Harvard College, Cambridge, MA (US)

(72) Inventors: Andrew Kruse, Roslindale, MA (US); Sarah Cecilia Erlandson, Georgetown, MA (US)

(21) Appl. No.: 17/998,057

(22) PCT Filed: May 7, 2021
(86) PCT No.: PCT/US2021/031260

§ 371 Date: Nov. 7, 2022

Related U.S. Application Data
(60) Provisional application No. 63/021,814, filed on May 8, 2020.

(51) Int. Cl. C07K 14/64; A61K 38/22; C12N 15/62; C12N 15/63

(52) U.S. Cl. CPC C07K 14/64; A61K 38/2221; C12N 15/62; C12N 15/63; C07K 2319/50

(57) ABSTRACT

The present invention provides novel recombinant relaxin-2 compositions and methods for making the same. Also disclosed herein are methods of treating relaxin-2-associated disorders or diseases using the compositions of the invention.

### CROSS-REFERENCE TO RELATED APPLICATIONS

This application claims the benefit of priority to U.S. Provisional Application No. 63/021,814, filed on May 8, 2020, the entire contents of which are expressly incorporated herein by reference.

### SEQUENCE LISTING

The instant application contains a Sequence Listing which has been submitted electronically in ASCII format and is hereby incorporated by reference in its entirety. Said ASCII copy, created on May 6, 2021, is named 117823-19620_SL.txt and is 79,204 bytes in size.

### TECHNICAL FIELD

The present invention relates to compositions and methods for modulating relaxin-2 activity.

### BACKGROUND OF THE INVENTION

Relaxins are small protein hormones distantly related to insulin. They regulate a variety of biological functions through their four receptors, RXFP1, RXFP2, RXFP3, and RXFP4. The first of these, RXFP1, has attracted particular interest as a therapeutic target due to its antifibrotic effects and its ability to enhance cardiac output. Its ligand, relaxin-2, has been evaluated in large scale clinical trials for the treatment of heart failure. Relaxin receptors may also be effective targets for the treatment of pulmonary arterial hypertension and various fibrotic diseases.

Both relaxins and their receptors are biochemically intractable molecules. Relaxins are composed of two chemically distinct chains, and existing methods for their production are slow, costly, and laborious. In addition, relaxin-2 produced using currently available methods has a short in vivo half-life. Accordingly, there is need in the art for recombinant relaxin-2 proteins that have a high level of biological activity, long circulating half-life, and are cost-effective to produce.

### SUMMARY OF THE INVENTION

Disclosed herein are novel relaxin-2 compositions and methods of use thereof for modulating, e.g., enhancing, relaxin-2 activity in a subject, e.g., a human subject. The composition and methods disclosed herein provide a means to treat and/or prevent relaxin associated diseases in a subject, such as a subject who can benefit from a modulated, e.g., increased or decreased, level of relaxin-2.

The compositions and methods disclosed herein are particularly advantageous in that they employ various fusion proteins and polypeptides disclosed herein that provide superior properties. For example, the fusion proteins and polypeptides of the present invention have improved pharmacokinetics, e.g., longer circulating half-life, or improved activity, e.g., enhanced activation of RXFP1 as compared to a native relaxin-2 protein. The fusion proteins and polypeptides of the present invention have been shown to provide improved activation of RXFP1 on a cell, with an EC₅₀ of about 0.085 nM to about 465 nM; and exhibit enhanced circulating half-life of at least about 77.5 hours to at least about 130 hours.

Accordingly, in one aspect, the present invention features a fusion protein. The fusion protein comprises, from N-terminus to C-terminus, a first peptide comprising an amino acid sequence that is at least about 85%, about 90%, about 91%, about 92%, about 93%, about 94%, about 95%, about 96%, about 97%, about 98%, about 99%, or about 100% identical to the entire amino acid sequence of SEQ ID NO: 2; a peptide linker comprising an amino acid sequence that is at least about 85%, about 90%, about 91%, about 92%, about 93%, about 94%, about 95%, about 96%, about 97%, about 98%, about 99%, or about 100% identical to the entire amino acid sequence of an amino acid sequence selected from the group consisting of DAASSHSHSSAR (SEQ ID NO: 14) and DAAGANANAGAR (SEQ ID NO: 16); and a second peptide comprising an amino acid sequence that is at least about 85% identical to the entire amino acid sequence of SEQ ID NO: 1; wherein the first peptide, the peptide linker, and the second peptide are operably linked.

In one embodiment, the fusion protein has an activity of a native relaxin-2 protein. In another embodiment, the fusion protein has at least about 50% activity of native relaxin-2 protein. In still another embodiment, the fusion protein has at least about 90% activity of native relaxin-2 protein. In yet another embodiment, the fusion protein has at least about 100% activity of native relaxin-2 protein. In one embodiment, the fusion protein has at least about 150% activity of native relaxin-2 protein.

In another embodiment, the peptide linker comprises an amino acid sequence that is at least about 90%, about 91%, about 92%, about 93%, about 94%, about 95%, about 96%, about 97%, about 98%, about 99%, or about 100% identical to the entire amino acid sequence of an amino acid sequence selected from the group consisting of DAASSHSHSSAR (SEQ ID NO: 14), DAASSHSHSSAA (SEQ ID NO: 15), and DAAGANANAGAR (SEQ ID NO: 16). In still another embodiment, the peptide linker comprises the amino acid sequence of DAASSHSHSSAR (SEQ ID NO: 14), DAASSHSHSSAA (SEQ ID NO: 15), or DAAGANANAGAR (SEQ ID NO: 16).

In still another embodiment, the first peptide has an amino acid sequence that is at least about 95%, about 96%, about 97%, about 98%, about 99%, or about 100% identical to the entire amino acid sequence of SEQ ID NO: 2, and the second peptide has an amino acid sequence that is at least about 95% identical to the entire amino acid sequence of SEQ ID NO: 1, wherein the fusion protein has a native relaxin-2 activity. In yet another embodiment, the amino acid sequence of the first peptide is selected from the group consisting of SEQ ID NOs: 2, 7, 8, 9, and 10, and wherein the amino acid sequence of the second peptide is selected from the group consisting of SEQ ID NOs: 1 and 6. In one embodiment, wherein the first peptide comprises a substitution selected from the group consisting of M4K, M25K, W28A, and combinations thereof. In another embodiment, the first peptide comprises the substitutions M4K, M25K, and W28A.

In another aspect, the present invention provides a fusion protein. The fusion protein comprises a first peptide, a peptide linker, and a second peptide, wherein the amino acid sequence of the fusion protein is at least about 85% identical to the entire amino acid sequence of an amino acid sequence selected from the group consisting of SEQ ID NOs: 47, 48, 49, 50, 51, 52, 53, 54, and 55.

In still another aspect, the present invention provides a fusion protein. The fusion protein comprises a first peptide, a peptide linker, and a second peptide, wherein the amino acid sequence of the fusion protein is at least about 85% identical to the entire amino acid sequence set forth in SEQ ID NO: 55. In one embodiment, the amino acid sequence of the fusion protein is set forth in SEQ ID NO: 55.

In various embodiments of the above aspects or any other aspect of the invention delineated herein, the fusion protein further comprises a first detectable label. In one embodiment, the first detectable label is operably linked to the N-terminus of the first peptide or the C-terminus of the second peptide. In another embodiment, the first detectable label is an immunoglobulin G (IgG) Fc peptide comprising an amino acid sequence that is at least about 85% identical to the entire amino acid sequence of SEQ ID NO: 20. In still another embodiment, the first detectable label has an amino acid sequence of SEQ ID NO: 20 or 21. In yet another embodiment, the first detectable label is operably linked to the N-terminus of the first peptide.

In one embodiment, the fusion protein further comprises a second linker, wherein the second linker is operably linked to the C-terminus of the first detectable label and to the N-terminus. In another embodiment, the second linker is selected from the group consisting of Gly-Gly-Ser, Ala-Ala-Ala, Pro-Pro-Pro, Gly-Ser-Gly, (Gly-Ser-Gly)₂ (SEQ ID NO: 57) and (Gly-Gly-Ser)₄ (SEQ ID NO: 17).

In one embodiment, the fusion protein has an in vivo circulating half-life of greater than about 10 hours. In another embodiment, the fusion protein has an in vivo circulating half-life of about 130 hours.

In another embodiment, the first detectable label is a polyhistidine tag having an amino acid sequence comprising an amino acid sequence that is at least about 85%, about 90%, about 91%, about 92%, about 93%, about 94%, about 95%, about 96%, about 97%, about 98%, about 99%, or about 100% identical to the entire amino acid sequence selected from the group consisting of SEQ ID NOs: 18 and 19. In still another embodiment, the first detectable agent comprises the amino acid sequence of SEQ ID NO: 18 or 19.

In one embodiment, the fusion protein further comprises a second detectable label. In another embodiment, the first detectable label is operably linked to the N-terminus of the first peptide, and the second detectable label is operably linked to the C-terminus of the second peptide. In still another embodiment, the first detectable label and the second detectable label are operably linked to the N-terminus of the first peptide. In yet another embodiment, the first detectable label and the second detectable label are different peptides.

In various embodiments of the above aspects, the fusion protein further comprises a cleavable linker. In one embodiment, the cleavable linker is a peptide subject to the specific digestion of a protease. In another embodiment, the protease is HRV 3C protease or thrombin. In still another embodiment, the cleavable linker is a peptide having the sequence of SEQ ID NO: 23, or a variant thereof.

In various embodiments of the above aspects, the fusion protein further comprises a signal peptide at the N-terminus of the fusion protein.

In one aspect, the present invention provides a fusion protein. The fusion protein includes a detectable label, a second linker, a first peptide, a peptide linker, and a second peptide, wherein the amino acid sequence of the fusion protein is at least about 85% identical to the entire amino acid sequence set forth in SEQ ID NO: 41, SEQ ID NO: 60, or SEQ ID NO: 61. In one embodiment, the amino acid sequence of the fusion protein is set forth in SEQ ID NO: 41, SEQ ID NO: 60, or SEQ ID NO: 61.

In one aspect, the present invention provides a fusion protein. The fusion protein includes a detectable label, a second linker, a first peptide, a peptide linker, and a second peptide, wherein the amino acid sequence of the fusion protein is at least about 85% identical to the entire amino acid sequence set forth in SEQ ID NO: 41.

In another aspect, the present invention provides a peptide linker comprising an amino acid sequence having at least about 85% amino acid identity to the entire amino acid sequence of an amino acid selected from the group consisting of DAASSHSHSSAR (SEQ ID NO: 14) and DAAGANANAGAR (SEQ ID NO: 16).

In another aspect, the present invention provides a fusion protein which includes, comprising from N-terminus to C-terminus: a first peptide comprising a relaxin B amino acid sequence; a peptide linker; and a second peptide comprising a relaxin A amino acid sequence, wherein the fusion protein has an activity of a native relaxin-2 protein, and wherein the fusion protein has a property selected from the group consisting of: (i) activates the relaxin-2 receptor RXFP1 on a cell surface with EC₅₀ of about 4.2 nM or less; (ii) a melting temperature of at least about 57° C.; (iii) a circulating half-life of at least about 77.5 hours; and (iv) any combination thereof.

In another aspect, the present invention provides a fusion protein comprising, from N-terminus to C-terminus, a first peptide comprising an amino acid sequence that is at least about 90% identical to the entire amino acid sequence of SEQ ID NO:10; a peptide linker; and a second peptide comprising a relaxin A amino acid sequence.

In one embodiment, the amino acid in the first peptide corresponding to amino acid 4 of SEQ ID NO:10 is K; the amino acid in the first peptide corresponding to amino acid 25 of SEQ ID NO:10 is K; and the amino acid in the first peptide corresponding to amino acid 28 of SEQ ID NO:10 is A.

In another embodiment, the peptide linker comprises the amino acid sequence of SEQ ID NO:16.

In another aspect, the present invention provides a fusion protein comprising, from N-terminus to C-terminus, a first peptide comprising a relaxin B amino acid sequence; a peptide linker comprising the amino acid sequence of SEQ ID NO:16; and a second peptide comprising a relaxin A amino acid sequence.

In one embodiment, the first peptide comprises the amino acid sequence of SEQ ID NO:10; the amino acid sequence of the peptide linker consists of the amino acid sequence of SEQ ID NO:16; the second peptide comprises an amino acid sequence that is at least about 85% identical to the entire amino acid sequence of SEQ ID NO:1; the second peptide comprises the amino acid sequence of SEQ ID NO:1; the first peptide comprises an amino acid sequence that is at least about 90% identical to the entire amino acid sequence of SEQ ID NO:10; the peptide linker comprises the amino acid sequence of SEQ ID NO:16; and the second peptide comprises an amino acid sequence that is at least about 85% identical to the entire amino acid sequence of SEQ ID NO:1, or the first peptide comprises the amino acid sequence of SEQ ID NO:10; the peptide linker comprises the amino acid sequence of SEQ ID NO:16; and the second peptide comprises the amino acid sequence SEQ ID NO:1.

In another aspect, the present invention provides a polypeptide comprising an amino acid sequence that is at least about 90% identical to the entire amino acid sequence of SEQ ID NO:10.

In one embodiment, the amino acid corresponding to amino acid 4 of SEQ ID NO:10 is K; the amino acid corresponding to amino acid 25 of SEQ ID NO:10 is K; and the amino acid corresponding to amino acid 28 of SEQ ID NO:10 is A.

In another embodiment, the polypeptide comprises an amino acid sequence comprising SEQ ID NO:10. In yet another embodiment, the amino acid sequence of the polypeptide consists of SEQ ID NO:10.

In yet another embodiment, the polypeptide further comprises an amino acid sequence that is at least about 85% identical to the entire amino acid sequence of SEQ ID NO:16. In yet another embodiment, the polypeptide further comprises an amino acid sequence comprising SEQ ID NO:16.

In yet another embodiment, the polypeptide further comprises an amino acid sequence that is at least about 85% identical to the entire amino acid sequence of SEQ ID NO:1. In yet another embodiment, the polypeptide further comprises an amino acid sequence comprising SEQ ID NO:1.

In another aspect, the present invention provides a polypeptide comprising an amino acid sequence that comprises SEQ ID NO:1 and SEQ ID NO:16.

In another aspect, the present invention provides a polypeptide comprising an amino acid sequence that comprises the amino acid sequences of SEQ ID NO:1, SEQ ID NO:10, and SEQ ID NO:16, wherein the amino acid sequence of SEQ ID NO:16 is interposed between the amino acid sequences of SEQ ID NO:1 and SEQ ID NO:10.

In still another aspect, the present invention provides a polynucleotide comprising a nucleotide sequence encoding the fusion protein or polypeptide of any embodiments of the above aspects. In one embodiment, the polynucleotide is an RNA molecule.

In yet another aspect, the present invention provides an expression vector. The expression vector comprises the polynucleotide of the above aspects. In one embodiment, the expression vector is a plasmid. In another embodiment, the expression vector is a viral vector.

In one aspect, the present invention provides a recombinant cell. The recombinant cell comprises the polynucleotide or the expression vector of the above aspects. In one embodiment, the cell is a prokaryotic cell or a eukaryotic cell. In another embodiment, the cell is a prokaryotic cell selected from the group consisting of E. coli cell and Bacillus cell. In still another embodiment, the cell is a eukaryotic cell selected from the group consisting of yeast cell, insect cell, and mammalian cell. In yet another embodiment, the cell is a mammalian cell selected from the group consisting of CHO cell, HeLa cell, and 293 cell. In one embodiment, the cell is an Expi293 cell.

In one aspect, the present invention provides a method of producing the fusion protein of the above aspects, comprising culturing the recombinant cell of the above aspects, and purifying the fusion protein.

In another aspect, the present invention provides a pharmaceutic composition. The pharmaceutic composition comprises an effective amount of the fusion protein of any one of the above aspects, or the polynucleotide of any one of the above aspects, or the expression vector of any one of the above aspects.

In still another aspect, the present invention provides a method of enhancing a relaxin-2-related activity in a cell, comprising contacting the cell with the fusion protein of any of the above aspects, thereby enhancing relaxin-2-related activity in the cell. In one embodiment, the fusion protein activates the relaxin-2 receptor, RXFP1, on a cell surface. In another embodiment, the method elevates cAMP levels in the cell, inducing vasodilation, inducing the expression of angiogenic factors, inducing the expression of MMPs, and inducing collagen degradation. In still another embodiment, the cell is selected from the group consisting of endothelial cells, vascular smooth muscle cells, other vascular cells, cardiomyocytes, other cardiac cells, and fibroblasts.

In one embodiment, the cell is within a subject. In another embodiment, the subject has a relaxin-2-associated disorder. In still another embodiment, the relaxin-2-associated disorder is selected from the group consisting of kidney diseases, fibrotic diseases, and cardiovascular diseases. In yet another embodiment, the disorder is selected from the group consisting of focal segmental glomerular sclerosis (FSGS), diabetic nephropathy, hepatorenal syndrome, scleroderma, idiopathic pulmonary fibrosis, renal fibrosis, cardiac fibrosis, NASH, dilated cardiomyopathy, diastolic heart failure, pulmonary arterial hypertension, chronic heart failure, acute heart failure, congestive heart failure, coronary artery disease, hypertension, and pre-eclampsia.

In one aspect, the present invention provides a method of treating a relaxin-2-associated disorder in a subject in need thereof. The method comprises administering to the subject an effective amount of the fusion protein of any one of the above aspects, the polynucleotide of any one of above aspects, the expression vector of any one of above aspects, or the pharmaceutical composition of above aspects, thereby treating the relaxin-2-associated disorder. In one embodiment, the relaxin-2-associated disorder is selected from the group consisting of kidney diseases, fibrotic diseases, and cardiovascular diseases. In another embodiment, the disorder is selected from the group consisting of focal segmental glomerular sclerosis (FSGS), diabetic nephropathy, hepatorenal syndrome, scleroderma, idiopathic pulmonary fibrosis, renal fibrosis, cardiac fibrosis, NASH, dilated cardiomyopathy, diastolic heart failure, pulmonary arterial hypertension, chronic heart failure, acute heart failure, congestive heart failure, coronary artery disease, hypertension, and pre-eclampsia. In still another embodiment, the method decreases arterial pressure, increases renal artery blood flow, increases cardiac filling at diastole, resolves established fibrosis, or suppresses new fibrosis development.

In another aspect, the present invention provides a kit. The kit comprises an effective amount of the fusion protein of any one of the above aspects, the polynucleotide of any one of the above aspects, the expression vector of any one of the above aspects, or the pharmaceutical composition of any one of the above aspects, and an instruction of use.

### BRIEF DESCRIPTION OF THE DRAWINGS

FIGS. 1A-1C are images showing electrophoresis and Coomassie blue staining of recombinant relaxin-2 proteins, SE001, SE201, SE202, SE203, SE204, SE205, SE206, SE207, and SE301.

FIG. 2 is a graph showing the size exclusion chromatography of SE301.

FIG. 3 is a graph showing the determination of Tₘ of SE301 using differential scanning fluorimetry.

FIG. 4 is a graph showing the activity of two recombinant relaxin-2 proteins, SE001 and SE004, as compared to native relaxin-2.

FIG. 5 is a graph showing the activity of three recombinant relaxin-2 proteins, SE101, SE102, and SE103, as compared to native relaxin-2.

FIG. 6 is a graph showing the activity of three recombinant relaxin-2 proteins, SE201, SE202, and SE203, as compared to native relaxin-2.

FIG. 7 is a graph showing the activity of four recombinant relaxin-2 proteins, SE204, SE205, SE206, and SE207, as compared to native relaxin-2.

FIG. 8 is a graph showing the activity of one recombinant relaxin-2 protein, SE301, as compared to native relaxin-2.

FIG. 9 is a graph showing the activity of one recombinant relaxin-2 protein, SE302, as compared to native relaxin-2.

FIG. 10 is a graph showing the activity of two recombinant relaxin-2 proteins, SE303 and SE304, as compared to native relaxin-2.

FIG. 11 is a graph showing the activity of one recombinant relaxin-2 protein, SE305, as compared to native relaxin-2.

FIG. 12 is a graph showing the activity of one recombinant relaxin-2 protein, SE401, as compared to native relaxin-2.

FIG. 13 is a graph showing the pharmacokinetics data for SE301.

FIG. 14 is a graph showing the activity of two recombinant relaxin-2 proteins, SE501 and SE502, as compared to native relaxin-2.

FIG. 15 is a graph showing the flow cytometry data for SE301.

### DETAILED DESCRIPTION OF THE INVENTION

The present invention is based upon, at least partly, the discovery that a recombinant single chain relaxin-2 protein, e.g., a fusion protein comprising a relaxin B chain, a linker, and a relaxin A chain, maintains a high level of biological activity as compared to a native relaxin-2. In some embodiments, the recombinant single chain relaxin-2 protein comprises an immunoglobulin G constant region (Fc domain) operably linked thereto with little or no impairment of biological activity. Accordingly, disclosed herein are novel recombinant relaxin-2 compositions and methods for making the same. Also disclosed herein are methods of treating relaxin-2-associated disorders or diseases using the compositions of the invention. The recombinant single chain relaxin-2 proteins according to the present invention possess several superior properties. For example, the recombinant single chain relaxin-2 proteins have improved pharmacokinetics, e.g., longer circulating half-life, or improved activity, e.g., enhanced maximum activation of RXFP1. It is also straightforward and cost-effective to produce the recombinant single chain relaxin-2 proteins according to the present invention.

### I. Definitions

In order that the present invention may be more readily understood, certain terms are first defined.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “about” or “approximately” means within 5%, or more preferably within 1%, of a given value or range.

As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.

“Therapeutically effective amount,” as used herein, is intended to include the amount of an agent or composition that, when administered to a patient for treating a subject having a relaxin-2-associated disease, is sufficient to effect treatment of the disease (e.g., by diminishing, ameliorating, or maintaining the existing disease or one or more symptoms of disease or its related comorbidities). The “therapeutically effective amount” may vary depending on the agent or composition, how it is administered, the disease and its severity and the history, age, weight, family history, genetic makeup, stage of pathological processes mediated by relaxin-2, the types of preceding or concomitant treatments, if any, and other individual characteristics of the patient to be treated.

Generally, the term “treatment” or “treating” is defined as the application or administration of a therapeutic agent to a patient, or application or administration of a therapeutic agent to an isolated tissue or cell line from a patient, said patient having a disease, a symptom of disease or a predisposition toward a disease, with the purpose to cure, heal, alleviate, relieve, alter, remedy, ameliorate, improve or affect the disease, the symptoms of disease or the predisposition toward disease. Thus, treating can include suppressing, inhibiting, preventing, treating, or a combination thereof. Treating refers, inter alia, to increasing time to sustained progression, expediting remission, inducing remission, augmenting remission, speeding recovery, increasing efficacy of/or decreasing resistance to alternative therapeutics, or a combination thereof.

“Suppressing” or “inhibiting,” refers, inter alia, to delaying the onset of symptoms, preventing relapse to a disease, decreasing the number or frequency of relapse episodes, increasing latency between symptomatic episodes, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment the symptoms are primary, while in another embodiment, symptoms are secondary.

“Primary” refers to a symptom that is a direct result of a disorder, e.g., diabetes, while, secondary refers to a symptom that is derived from or consequent to a primary cause. Symptoms may be any manifestation of a disease or pathological condition.

Accordingly, as used herein, the term “treatment” or “treating” includes any administration of a composition described herein and includes: (i) preventing the disease from occurring in a subject which may be predisposed to the disease but does not yet experience or display the pathology or symptomatology of the disease; (ii) inhibiting the disease in an subject that is experiencing or displaying the pathology or symptomatology of the diseased (i.e., arresting further development of the pathology and/or symptomatology); or (iii) ameliorating the disease in a subject that is experiencing or displaying the pathology or symptomatology of the diseased (i.e., reversing the pathology and/or symptomatology).

By “treatment,” “prevention” or “amelioration” of a disease or disorder is meant delaying or preventing the onset of such a disease or disorder, reversing, alleviating, ameliorating, inhibiting, slowing down or stopping the progression, aggravation or deterioration the progression, or severity of a condition associated with such a disease or disorder. In one embodiment, the symptoms of a disease or disorder are alleviated by at least 5%, at least 10%, at least 20%, at least 30%, at least 40%, or at least 50%.

Efficacy of treatment is determined in association with any known method for diagnosing the disorder. Alleviation of one or more symptoms of the disorder indicates that the composition confers a clinical benefit. Any of the therapeutic methods described above can be applied to any suitable subject including, for example, mammals such as dogs, cats, cows, horses, rabbits, monkeys, and most preferably, humans.

As used herein, the term “subject” includes any subject who may benefit from being administered a hydrogel or an implantable drug delivery device of the invention. The term “subject” includes animals, e.g., vertebrates, amphibians, fish, mammals, non-human animals, including humans and primates, such as chimpanzees, monkeys, and the like. In one embodiment of the invention, the subject is a human.

The term “subject” also includes agriculturally productive livestock, for example, cattle, sheep, goats, horses, pigs, donkeys, camels, buffalo, rabbits, chickens, turkeys, ducks, geese, and bees; and domestic pets, for example, dogs, cats, caged birds and aquarium fish, and also so-called test animals, for example, hamsters, guinea pigs, rats, and mice.

### II. Compositions of the Invention

A. Relaxin-2

Human relaxin-2 is a peptide hormone with multiple pleiotropic actions. Initially thought to be only a reproductive hormone involved in facilitating delivery of a baby, more recent studies have demonstrated that relaxin-2 plays a key role in inflammatory and matrix remodeling processes and possesses potent vasodilatory, angiogenic, and other cardioprotective actions. The vasodilatory effects of relaxin-2 are thought to involve promotion of nitric oxide and the gelatinases, matrix metalloproteinase-2 and matrix metalloproteinase-9, in addition to antagonism of the vasoconstricting actions of endothelin-1 and angiotensin II. This causes systemic and renal vasodilation, increased arterial compliance, and other vascular changes. These findings have led to evaluation of relaxin-2 as drug for the treatment of patients with acute heart failure (AHF) and other diseases. Furthermore, the matrix remodeling actions of relaxin-2 have enhanced its reputation as a rapidly acting but safe antifibrotic agent, which has been further supported by its ability to successfully inhibit and/or reverse fibrosis in every preclinical model of experimental disease evaluated to date.

The actions of relaxin-2 are thought to be mediated through its native receptor RXFP1 (originally named LGR7), which is a leucine-rich repeat containing G-protein coupled receptor that is characterized by an unusually large ectodomain. Human relaxin-2 can also bind to and activate the related receptor, RXFP2, which is the native receptor for insulin-like peptide 3 (INSL3), suggesting that potential cross-reactivity may be associated with its diverse actions.

Native relaxin-2 has an insulin-like core structure containing two chains (relaxin A and relaxin B) and three disulfide bonds. As used herein, the term “native relaxin-2” refers to any relaxin-2, e.g., human relaxin-2, that is naturally produced in a subject. The naturally occurring orthologs of human relaxin-2, such as mouse relaxin-1, are also contemplated as native relaxin-2 of the invention. Native relaxin-2 also includes the relaxin-2 produced using any recombinant methods and has substantially the same structure, i.e., primary structure, secondary structure, and tertiary structure, and substantially same biological activity, e.g., binding to RXFP1, to a naturally occurring relaxin-2.

Human relaxin A and B chains are derived from a single gene product (GenBank Accession No. CAA25460.1). The human precursor relaxin-2 protein is normally proteolyzed after translation, leading to the mature A/B form. In some embodiments, an exemplary human native relaxin-A has the amino acid sequence as set forth in SEQ ID NO: 1 (QLYSALANKCCHVGCTKRSLARFC). In some embodiments, an exemplary human native relaxin-B has the amino acid sequence as set forth in SEQ ID NO: 2 (DSWMEEVIKLCGRELVRAQIAICGMSTWS). The mouse equivalent of human relaxin-2 is murine relaxin-1, similarly derived from a precursor protein (GenBank Accession No. CAA81611.1). In some embodiments, an exemplary mouse native relaxin-A has the amino acid sequence as set forth in SEQ ID NO: 3 (ESGGLMSQQCCHVGCSRRSIAKLYC). In some embodiment, an exemplary mouse native relaxin-B has the amino acid sequence as set forth in SEQ ID NO: 4 (RVSEEWMDGFIRMCGREYARELIKICGASVGRLAL).

B. Recombinant Relaxin-2

1. Relaxin A and Relaxin B

The present invention provides recombinant relaxin-2 proteins, e.g., recombinant human relaxin-2, that have a high level of biological activity compared to native relaxin-2 while allowing modification for enhanced serum half-life. The term “recombinant” indicates that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well-known molecular biological procedures. A “recombinant DNA molecule,” is comprised of segments of DNA joined together by means of such molecular biological techniques. The term “recombinant protein” or “recombinant polypeptide” as used herein refers to a protein molecule which is expressed using a recombinant DNA molecule. A “recombinant host cell” is a cell that contains and/or expresses a recombinant nucleic acid. The term recombinant relaxin-2 and engineered relaxin-2 can be used interchangeably.

The recombinant relaxin-2 proteins comprise a native relaxin A, e.g., human relaxin A, or a variant thereof, and a native relaxin B, e.g., human relaxin B, or a variant thereof. As used herein, “relaxin A,” “relaxin B,” “relaxin-2,” and other proteins or peptides, refer to the native or variant protein or peptide when the name of the protein or peptide is used independently from the term “native” or “variant.” The term “variant,” as used herein, refers to a protein or peptide derived from one or more amino acid insertion, substitution, or deletion from a precursor protein or peptide (e.g., “parent” protein or peptide). In certain embodiments, the variant comprises at least one modification that includes a change in charge compared to the precursor protein or peptide. In certain preferred embodiments, the precursor protein or peptide is a parent protein or peptide that is a native or peptide.

In certain embodiments, a variant protein or peptide, e.g., a variant human relaxin-A or relaxin-B, has at least about 85% sequence identity, e.g., about 90%, about 91%, about 92%, about 93%, about 94%, about 95%, about 96%, about 97%, about 98%, or about 99%, to the native protein or peptide. The term “sequence identity,” as used herein, refers to a comparison between pairs of nucleic acid or amino acid molecules, i.e., the relatedness between two amino acid sequences or between two nucleotide sequences. In general, the sequences are aligned so that the highest order match is obtained. Methods for determining sequence identity are known and can be determined by commercially available computer programs that can calculate the percentage of identity between two or more sequences. A typical example of such a computer program is CLUSTAL. As an illustration, by a polynucleotide having a nucleotide sequence having at least, for example, 90% sequence identity to a reference nucleotide sequence is intended that the nucleotide sequence of the polynucleotide is identical to the reference sequence except that the polynucleotide sequence may include on average of up to 10 point mutations per each 100 nucleotides of the reference nucleotide sequence. In other words, to obtain a polynucleotide having a nucleotide sequence at least 90% identical to a reference nucleotide sequence, up to 10% of the nucleotides in the reference sequence may be deleted or substituted with another nucleotide, or a number of nucleotides up to 10% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Similarly, by a polypeptide having an amino acid sequence having at least, for example, 90% sequence identity to a reference amino acid sequence, it is intended that the amino acid sequence of the polypeptide is identical to the reference sequence except that the polypeptide sequence may include on average up to 10 amino acid alterations per each 100 amino acids of the reference amino acid. In other words, to obtain a polypeptide having an amino acid sequence at least 90% identical to a reference amino acid sequence, up to 10% of the amino acid residues in the reference sequence may be deleted or substituted with another amino acid, or a number of amino acids up to 10% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

Preferred methods to determine identity are designed to give the largest match between the sequences tested. Methods to determine identity are described in publicly available computer programs. Preferred computer program methods to determine identity between two sequences include the GCG program package, including GAP (Devereux et al., 1984, Nucl. Acid. Res. 12: 387; Genetics Computer Group, University of Wisconsin, Madison, Wis., USA), BLASTP, BLASTN, and FASTA (Altschul et al., 1990, J. Mol. Biol. 215: 403-410). The BLASTX program is publicly available from the National Center for Biotechnology Information (NCBI) and other sources (BLAST Manual, Altschul et al. NCB/NLM/NIH Bethesda, Md., USA; Altschul et al., supra). The well-known Smith Waterman algorithm may also be used to determine identity. For example, using the computer algorithm GAP (Genetics Computer Group, University of Wisconsin, Madison, Wis., USA), two proteins for which the percent sequence identity is to be determined are aligned for optimal matching of their respective amino acids (the “matched span,” as determined by the algorithm). A gap opening penalty (which is calculated as 3 times the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix is also used by the algorithm (see Dayhoff et al., 1978, Atlas of Protein Sequence and Structure, Vol. 5, Suppl. 3, (1978) for the PAM 250 comparison matrix; Henikoff et al., 1992, Proc. Natl. Acad. Sci USA 89: 10915-10919, for the BLOSUM 62 comparison matrix).

In certain embodiments, a variant human relaxin A comprises a substitution Q1D (SEQ ID NO: 5). As used herein, the format “L₁NL₂” represents a substitution at location N. “L₁” is a single letter symbol that represents the amino acid at location N of a native protein or peptide. “N” is a number that represents the location of the substitution, counting from the first amino acid of a native protein or peptide, e.g., the first amino acid of human native relaxin A having the sequence set forth in SEQ ID NO: 1, or the first amino acid of human native relaxin B having the sequence set forth in SEQ ID: 2. “L₂” is a single letter symbol that represents the amino acid that replaces L₁.

In certain embodiments, a variant human relaxin B comprises a truncated peptide (SEQ ID NO: 6), in which the first five amino acids (DSWME (SEQ ID NO: 59)) were deleted from the human native relaxin B. In certain embodiments, a variant human relaxin B comprises one or more substitutions selected from the group consisting of M4K, R13A, R13D, R17A, R17D, I20A, I20D, M25K, and W28A.

In certain embodiments, the variant human relaxin B is selected from the group consisting of SEQ ID NOs: 7, 8, 9, and 10.

In some embodiments, the variant human relaxin B has the sequence set forth in SEQ ID NOs: 11, 12, and 13.

In some embodiments, the recombinant relaxin-2 protein is a single chain protein, e.g., a fusion protein. In the single chain relaxin-2 recombinant protein, the relaxin A and relaxin B of the recombinant relaxin-2 are operably linked, e.g., covalently linked, via a linker. The terms “operably linked”, “in operable combination”, and “in operable order” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. In certain embodiments, relaxin A, relaxin B, and the linker are covalently linked in the following operable order:

Relaxin B-Linker-Relaxin A.

In certain embodiments, the recombinant relaxin-2 comprises a linker having an amino acid sequence of DAASSHSHSSAR (SEQ ID NO: 14) or a variant thereof. In some embodiments, the linker has a sequence of DAASSHSHSSAA (SEQ ID NO: 15). In some embodiments, the recombinant relaxin-2 comprises a linker having an amino acid sequence of DAAGANANAGAR (SEQ ID NO: 16) or a variant thereof. The linker with amino acid sequence DAASSHSHSSAA (SEQ ID NO: 15) is reported in a publication on a method to produce native relaxin-3 (Luo et al., A simple approach for the preparation of mature human relaxin-3, Peptides, 2010).

2. Linker

In some embodiments, the recombinant relaxin-2 comprises a linker. The linker covalently links at least two components of the recombinant relaxin-2 in an operable order. The term “linker,” as used herein, refers to a chemical group or molecule that connects two molecules or moieties (e.g., two peptides such as relaxin A and relaxin B). Typically, a linker is placed between or flanked by two groups, molecules, or other moieties, connected to each other through covalent bonds, and hence the two connect. In some embodiments, the linker comprises one amino acid or multiple amino acids (e.g., a peptide or protein). In some embodiments, the linker comprises a cleavable site. For example, the linker includes a peptide that can be cleaved by HRV3C protease. In some embodiments, the linker is any stretch of amino acids and is at least 1, at least 2, at least 3, at least 4, at least 5, at least 6, at least 7, at least 8, at least 9, at least 10, at least 15,It has at least 20, at least 25, at least 30, at least 40, at least 50, or 51 or more amino acids.

In some embodiments, the peptide linker comprises a repeat (repeat) of the tripeptide Gly-Gly-Ser or a variant thereof, for example comprising the sequence (GGS)ₙ, where n is at least 1, 2, 3, 4, 5, 6, 7, Represents 8, 9, 10, or 11 or more repeats. In some embodiments, the linker comprises the sequence (GGS)₄ (SEQ ID NO: 17). In some embodiments, the peptide linker comprises a repeat (repeat) of the tripeptide Gly-Ser-Gly or a variant thereof, for example comprising the sequence (GSG)ₙ, where n is at least 1, 2, 3, 4, 5, 6, 7, Represents 8, 9, 10, or 11 or more repeats. In some embodiments, the linker comprises the sequence (GSG)₂ (SEQ ID NO: 57). In some embodiments, the peptide linker comprises a repeat (repeat) of the tripeptide Ala-Ala-Ala, for example comprising the sequence (AAA)ₙ, where n is at least 1, 2, 3, 4, 5, 6, 7, 8, 9, 10, or 11 or more repeats. In some embodiments, the peptide linker comprises a repeat (repeat) of the tripeptide Pro-Pro-Pro, for example comprising the sequence (PPP)ₙ, where n is at least 1, 2, 3, 4, 5, 6, 7, 8, 9, 10, or 11 or more repeats.

3. Detectable Label

In some embodiments, the recombinant relaxin-2 of the invention further comprises a detectable label such as an enzymatic, fluorescent, or affinity label to allow for detection and isolation of the protein. Such detectable labels may include, but are not limited to polyhistidine tags, immunoglobulin Fc tags, myc tags, HA tags, glutathione S-transferase, fluorescent tags, or variants thereof. Non-limiting examples of suitable enzymes include horseradish peroxidase, alkaline phosphatase, β-galactosidase, glucose oxidase, or acetylcholinesterase. In some embodiments, the detectable label is a polyhistidine tag, such as 6×His (SEQ ID NO: 18) or a variant thereof, or 8×His (SEQ ID NO: 19) or a variant thereof. In some embodiments, the detectable label can be used for antibody affinity chromatography or detection, e.g., Protein C tag. Examples of Protein C tags include, but are not limited to, a peptide having the amino acid sequence of EDQVDPRLIDGKGS (SEQ ID NO: 24), or a variant thereof.

In some embodiments, the detectable label is an immunoglobulin Fc domain, e.g., IgG1 Fc domain (SEQ ID NO: 20), or a variant thereof, e.g., IgG1 Fc domain comprising a N77Q substitution (SEQ ID NO: 21). The detectable label can also have other functions. For example, an immunoglobulin Fc domain tag may increase the half-life of the recombinant relaxin-2 in a subject. Fc fragments also promote immune effector functions including complement activation and cellular cytotoxicity via Fc gamma receptor binding. In some embodiments, the Fc fragment of the recombinant single chain relaxin-2 may include one or more substitutions to attenuate immune effector functions, e.g., a substitution of Asn297 by Gln in the IgG1 Fc region (referred to as N77Q in the recombinant relaxin-2 proteins). Exemplary effector function-attenuating substitutions of Fc fragment include, but are not limited to, N297G (NG) and D265A, N297G, L234A, L235A, and P329G. Exemplary effector function-attenuating substitutions are described in Lo et al., Effector-attenuating Substitutions That Maintain Antibody Stability and Reduce Toxicity in Mice, J. Biol. Chem., 292, 3900-08 (2017), incorporated herein by reference.

In certain embodiments, the detectable label is a serum albumin, e.g., a human serum albumin or mouse serum albumin. Examples of mouse serum albumin include a protein having an amino acid sequence of SEQ ID NO: 22, or a variant thereof.

The detectable label can be operably linked, e.g., covalently linked to the N-terminus or to the C-terminus of the relaxin A or the relaxin B. The detectable label can be operably linked directly to the relaxin A or the relaxin B. The detectable label can also be operably linked to the relaxin A or the relaxin B via a linker, such as a GGS or GSG linker.

The detectable label can also be operably linked to the relaxin A or the relaxin B via a cleavable linker, such as a peptide that is cleavable by a protease. Exemplary proteases that specifically cleave the cleavable linkers include, but are not limited to, thrombin, HRV3C protease, factor Xa, and TEV protease. Examples of HRV3C sites include, but are not limited to, a peptide having the amino acid sequence of LEVLFQGP (SEQ ID NO: 23), or a variant thereof, or GSLEVLFQGPG (SEQ ID NO: 58), or a variant thereof. Examples of thrombin sites include, but are not limited to, a peptide having the amino acid sequence LVPRGS (SEQ ID NO: 56), or a variant thereof.

4. Biological Activity of Recombinant Relaxin-2

In some embodiments, the recombinant relaxin-2 of the invention has high level of biological activity as compared to native relaxin-2. For example, the recombinant relaxin-2 may have about at least about 50% to about at least 2fold biological activity as compared to native relaxin-2. In some embodiments, the recombinant relaxin-2 has at least about 50%, about 80%, about 90%, about 100%, about 110%, about 120%, about 130%, about 140%, about 150%, about 160%, about 170%, about 180%, about 190%, or about 2fold biological activity as compared to native relaxin-2. In certain embodiments, the recombinant relaxin-2 has more than about 2fold biological activity as compared to native relaxin-2.

The biological activity can be any biological activity of native relaxin-2. For example, the biological activity can be the recombinant relaxin-2's capacity to bind the receptor of native relaxin-2, RXFP1. The binding of relaxin-2 to RXFP1 can be measured by any well-known methods in the art, such as radioligand binding. In some embodiments, the recombinant relaxin-2 binds to RXFP1 on the cell surface.

In some embodiments, the biological activity can be the recombinant relaxin-2's capacity to activate RXFP1 on the cell surface. Without wishing to be bound by any theory, the present invention is based upon, at least in part, the surprising discovery that some exemplary recombinant relaxin-2 proteins exhibit higher maximum activation of RXFP1 as compared to native two chain relaxin-2. The activation of RXFP1 by the recombinant relaxin-2 can be determined by the increase of cAMP using any methods well known in the art, such as measuring the activity of a cAMP-driven report gene, e.g., β-galactosidase. The activation of RXFP1 by recombinant relaxin-2 in a cell may also be determined by measuring the expression of certain genes, such as angiogenic factors, e.g., VEGF, or the expression of MMPs using well-known methods in the art. In some embodiments, the biological activity is a physiological, biochemical activity or any other effect-inducing activity of the relaxin-2. Exemplary biological activities include, but are not limited to, vasodilation, collagen degradation, angiogenesis, decreasing arterial blood pressure, increasing renal artery blood flow, increasing cardiac filling at diastole, resolving established fibrosis, and suppressing new fibrosis development.

In certain embodiments, the present invention provides proteins or peptides, e.g., recombinant relaxin-2, that have a high level of activity, e.g., at least about 50% to about 2fold biological activity of native relaxin-2, in one aspect, but has a low level of activity, e.g., less than about 50% biological activity of native relaxin-2, in another aspect. In some embodiments, the recombinant relaxin-2 has less than about 50%, about 40%, about 30%, about 20%, about 10%, or about 5% of biological activity in one aspect as compared to native relaxin-2. For example, a recombinant relaxin-2 may bind to a RXFP1 with high affinity, e.g., at least about 50% affinity to about 2fold as compared to native relaxin-2, but low activity in activating RXFP1, e.g., less than about 50% capacity in activate RXFP1. Such a recombinant relaxin-2 can be a dominant negative variant that reduces relaxin-2 activity in a subject in need thereof.

In some embodiments, the present invention provides proteins or peptides, e.g., recombinant relaxin-2, that have improved pharmacokinetics profiles. Without wishing to be bound by any theory, the present invention is based upon, at least in part, the surprising discovery that some exemplary recombinant relaxin-2 proteins exhibit much longer circulating half-life as compared to the native two chain relaxin-2. For example, the recombinant single chain relaxin-2 of the present invention may have a circulating half-life of greater than about 5 hours, e.g., greater than about 10 hours, greater than about 20 hours, greater than about 50 hours, greater than about 75 hours, greater than about 100 hours, greater than about 125 hours, or greater than about 150 hours. Values and ranges intermediate to the recited values are also intended to be part of this invention. In certain embodiments, the recombinant single chain relaxin-2 of the present invention has a circulating half-life of about 130 hours. Surprisingly, a single chain relaxin-2 of the present invention may have a longer circulating half-life than a native two chain relaxin-2. For example, the circulating half-life of a native two chain relaxin-2 may be less than about 5 hours. (See, e.g., Chen et al., The Pharmacokinetics of Recombinant Human Relaxin in Non-Pregnant Women after Intravenous, Intravaginal, and Intracervical Administration, Pharm. Res. 10: 834038 (1993), incorporated herein by reference).

“Circulating half-life,” as used herein, refers to the time it takes for the blood plasma concentration of a drug, e.g., a native relaxin-2 or a recombinant single chain relaxin-2, to halve its steady-state when circulating in the full blood of an organism. Circulating half-life of a particular agent may vary depending on a multitude of factors including, but not limited to, dosage, formulation, and/or administration route of the agent. One of ordinary skill in the art is able to determine the circulating half-life of an agent, e.g., a protein, e.g., a recombinant relaxin-2, using well known methods in the art, such as the method described in Example 3, or Chen supra.

5. Nucleic Acid Molecule Encoding Recombinant Relaxin-2

The invention also provides nucleic acid molecules that encode any of the protein or peptide, e.g., recombinant relaxin-2, described herein. In some embodiments, the nucleic acid molecules of the invention are a DNA molecule. In some embodiments, the nucleic acid molecules of the invention are an RNA molecule.

The individual strand or strands of a DNA molecule encoding any of the protein or peptide, e.g., recombinant relaxin-2, can be transcribed from a promoter in an expression vector. Where two separate proteins or peptides are to be expressed to generate, for example, a relaxin A and a relaxin B, two separate expression vectors can be co-introduced (e.g., by transfection or infection) into a target cell.

Expression vectors are generally DNA plasmids or viral vectors. Expression vectors compatible with eukaryotic cells, preferably those compatible with vertebrate cells, can be used to produce recombinant relaxin-2 as described herein. Production and purification of recombinant proteins are well known in the art, such as the methods described in “Molecular Cloning: A Laboratory Manual, Sambrook, et al. (1989) Cold Spring Harbor Laboratory Press. Without wishing to be bound by any theory, the present invention is based upon, at least in part, the surprising discovery that some exemplary recombinant relaxin-2 proteins can be produced in high yield (see, e.g., Example 3, Table 2).

The nucleic acids encoding a protein described herein, e.g., a recombinant relaxin-2, may be incorporated into a vector.

Expression of natural or synthetic nucleic acids is typically achieved by operably linking a nucleic acid encoding the gene of interest to a promoter, and incorporating the construct into an expression vector. The vectors can be suitable for replication and integration in eukaryotes. Typical cloning vectors contain transcription and translation terminators, initiation sequences, and promoters useful for expression of the desired nucleic acid sequence.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-la (EF-la). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Further, the present invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The expression vector can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Useful selectable markers include, for example, antibiotic-resistance genes, such as neo and the like.

Reporter genes may be used for identifying potentially transfected cells and for evaluating the functionality of transcriptional control sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of a reporter gene is identified as the promoter. Such promoter regions may be linked to a reporter gene and used to evaluate agents for the ability to modulate promoter-driven transcription.

In certain embodiment, the expression vectors are plasmid vectors, e.g., prokaryotic plasmid vectors or eukaryotic plasmid vectors. Exemplary prokaryotic plasmid vectors include, but are not limited to, pET expression series plasmid and pGEX expression series plasmid. Exemplary eukaryotic expression plasmids include, but are not limited to, yeast expression plasmid, plant cell expression plasmid, insect cell expression plasmid, avian cell expression plasmid, and mammalian expression plasmid. Exemplary mammalian expression plasmids include, but are not limited to, pRc/CMV, pcDNA3.1, pcDNA4, pcDNA6, pGene/V5, pFUSE-hIgG1-Fc2, pTT, and pED.dC. In certain embodiments, the expression plasmids comprise one or more inducible elements to control the expression of the recombinant single chain relaxin-2. Exemplary plasmids comprising inducible elements include, but are not limited to, pcDNA3.1-Zeo-tetO, a modified pcDNA3.1 plasmid for tetracycline-inducible protein expression and Zeocin antibiotic resistance.

In certain embodiments, the expression vectors of the invention can be delivered to a host cell for in vitro production of the protein or peptide, e.g., recombinant relaxin-2. The invention also provides a recombinant cell containing the nucleic acid molecule encoding any protein or peptide of the invention or a vector comprising such a nucleic acid molecule. Methods of introducing nucleic acid molecules into a cell are well known in the art, including, but not limited to, transformation, transfection, viral infection, or electroporation.

Examples of host cells include, but are not limited to, prokaryotic and eukaryotic cells selected from any of the Kingdoms of life. Examples of eukaryotic cells include, but are not limited to, protist, fungal, plant and animal cells. Non-limiting examples of host cells include, but are not limited to, the prokaryotic cell E. coli; mammalian cell lines CHO, HEK 293, HeLa, Expi293F, and COS; the insect cell line Spodoptera frugiperda cell line Sf9 and Trichoplusia ni cell line HighFive; and the fungal cell Saccharomyces cerevisiae.

In certain embodiments, the expression vectors can be used to deliver and/or express the gene encoding any protein or peptide of the invention to a cell in vivo for gene therapy. Vectors, including those derived from retroviruses such as lentiviruses, are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector. Viral vector technology is well known in the art and described in a variety of virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

Viral vector systems that can be utilized with the methods and compositions described herein include, but are not limited to, (a) adenovirus vectors; (b) retrovirus vectors, including but not limited to, lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g., canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g., EPV and EBV vectors. Constructs for the recombinant expression of a disrupting agent will generally require regulatory elements, e.g., promoters, enhancers, etc., to ensure the expression of the disrupting agent in target cells. Other aspects to consider for vectors and constructs are known in the art.

Methods of delivering viral vectors into cells in vivo are well known in the art. The viral vectors can be administered by any means known in the art including, but not limited to oral, intraperitoneal, or parenteral routes, including intracranial (e.g., intraventricular, intraparenchymal, and intrathecal), intravenous, intramuscular, subcutaneous, transdermal, airway (aerosol), nasal, rectal, and topical (including buccal and sublingual) administration.

The RNA molecules comprising the gene that encodes any protein or peptide of the invention can be used to deliver and/or express the gene in vivo for gene therapy. Methods for formulating and delivering the RNA molecules the gene in vivo are well known in the art, such as the methods described in U.S. Patent Publication 2016/0038612A1, incorporated herein by reference.

C. Pharmaceutical Composition and Administration

The present invention provides pharmaceutical compositions comprising the proteins or peptides, e.g., recombinant relaxin-2, or the nucleic acid molecules, or the expression vector of the present invention. The pharmaceutical compositions of the invention are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™, Life Technologies, Carlsbad, Calif.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of the proteins, peptides, or the nucleic acid molecules of the invention administered to a patient may vary depending upon the age and the size of the patient, target disease, conditions, route of administration, and the like. The preferred dose is typically calculated according to body weight or body surface area. Depending on the severity of the condition, the frequency and the duration of the treatment can be adjusted. Effective dosages and schedules for administering a recombinant relaxin-2 may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pens and autoinjector delivery devices have applications in the subcutaneous delivery of a pharmaceutical composition of the present invention. Examples include, but are not limited to, AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPT1PEN™, OPTIPEN PRO™, OPTIPEN STARLET™, and OPTICL1K™ (Sanofi-Aventis, Frankfurt, Germany), to name only a few. Examples of disposable pen delivery devices having applications in subcutaneous delivery of a pharmaceutical composition of the present invention include, but are not limited to, the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990,

Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending, or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

### III. Therapeutic Uses of Recombinant Relaxin-2

A. Methods of Using Recombinant Relaxin-2 The present invention includes methods comprising administering to a subject in need thereof a therapeutic composition comprising a recombinant relaxin-2 of the invention. The therapeutic composition can comprise any of the proteins or peptides as disclosed herein and a pharmaceutically acceptable carrier or diluent. As used herein, the expression “a subject in need thereof” means a human or non-human animal that exhibits one or more symptoms or indicia of a relaxin-2 associated disorder or disease, or who otherwise would benefit an increase or decrease in relaxin-2 activity. The proteins or peptides of the invention (and therapeutic compositions comprising the same) are useful, inter alia, for treating any disease or disorder in which activation or deactivation of RXFP1 is beneficial.

In certain embodiments, the present invention provides methods for activating RXFP1 on a cell surface, comprising administering an effective amount of the proteins or peptides of the invention, e.g., recombinant relaxin-2, to a subject in need thereof, thereby activating RXFP1 on the surface of the cell. Activation of RXFP1 on the cell surface can lead to cellular responses including, but not limited to, the elevation of cAMP levels, vasodilation, the expression of angiogenic factors including VEGF, the expression of MMPs, and collagen degradation. In some embodiments, the cell is selected from the group consisting of endothelial cells, vascular smooth muscle cells, other vascular cells, cardiomyocytes, other cardiac cells, and fibroblasts.

In some embodiments, the present invention provides methods for treat various relaxin-2 associated diseases. As used herein, the term “relaxin-2-associated disease,” is a disease or disorder that is caused by, or associated with, relaxin-2 protein production or relaxin-2 protein activity. The term “relaxin-2-associated disease” includes a disease, disorder or condition that would benefit from an increase in relaxin-2 protein activity. Non-limiting examples of relaxin-2-associated diseases include, for example, kidney diseases including but not limited to, focal segmental glomerular sclerosis (FSGS), diabetic nephropathy, hepatorenal syndrome; fibrotic diseases including but not limited to, scleroderma, idiopathic pulmonary fibrosis, renal fibrosis, cardiac fibrosis, NASH; cardiovascular diseases including dilated cardiomyopathy, diastolic heart failure, pulmonary arterial hypertension, chronic heart failure, acute heart failure, congestive heart failure, coronary artery disease, hypertension, pre-eclampsia. Further details regarding signs and symptoms of the various diseases or conditions are provided herein and are well known in the art.

Administration of the compositions according to the methods of the invention may result in a reduction of the severity, signs, symptoms, or markers of a relaxin-2-associated disease or disorder in a patient with a relaxin-2-associated disease or disorder. By “reduction” in this context is meant a statistically significant decrease in such level. The reduction (absolute reduction or reduction of the difference between the elevated level in the subject and a normal level) can be, for example, at least about 20%, 25%, 30%, 35%, 40%, 45%, 50%, 55%, 60%, 65%, 70%, 75%, 80%, 85%, 90%, or 95%, or to below the level of detection of the assay used.

B. Combination Therapies and Formulations

The present invention includes compositions and therapeutic formulations comprising any of the exemplary proteins or peptides, e.g., recombinant relaxin-2 protein, or nucleic acid molecules, described herein in combination with one or more additional therapeutically active components, and methods of treatment comprising administering such combinations to subjects in need thereof.

Exemplary additional therapeutic agents include any therapeutic agents that may be used for the treatment of any relaxin-2-related disorders described herein. Exemplary additional therapeutic agents that may be combined with or administered in combination with a recombinant relaxin-2 protein, or nucleic acid molecules, of the present invention include, but are not limited to, angiotensin II receptor blockers, e.g., azilsartan, candesartan, eprosartan, losartan, ACE inhibitors, e.g., lisinopril, benazepril, captopril, enalapril, moexipril, perindopril, quinapril, trandolapril, calcium channel blockers, e.g., amlodipine, amlodipine and benazepril, amlodipine and valsartan, diltiazem, felodipine, isradipine, nicardipine, nimodipine, nisoldipine, verapamil, or diuretics, e.g., chlorthalidone, hydrochlorothiazide, metolazone, indapamide, torsemide, furosemide, bumetanide, amiloride, triamterene, spironolactone, eplerenone, aldosterone antagonist, e.g., spironolactone, eplerenone, digoxin, e.g., lanoxin, beta blockers, e.g., carvedilol, metoprolol, bisoprilol.

In some embodiments, the additional therapeutic agents are drugs for fibrosis, including, but not limited to, small molecule drugs and antibodies. Exemplary anti-fibrosis drugs include, but are not limited to, TGF-β inhibitors, e.g., small molecules such as hydronidone, distiertide, or antibodies such as fresolimumab, PDGF or VEGF antagonist, e.g., small molecules such as imatinib, nilotinib, or any drugs that target extracellular factors that are involved in the pathogenesis of fibrosis. The description of exemplary drugs for fibrosis can be found, e.g., Li et al., Drugs and Targets in Fibrosis, Frontiers in Pharm., 8: Article 855 (2007), incorporated herein by reference.

The additional therapeutically active component(s) may be administered just prior to, concurrent with, or shortly after the administration of an antigen-binding molecule of the present invention; (for purposes of the present disclosure, such administration regimens are considered the administration of a recombinant relaxin-2 “in combination with” an additional therapeutically active component).

The present invention includes pharmaceutical compositions in which a recombinant relaxin-2 of the present invention is co-formulated with one or more of the additional therapeutically active component(s) as described elsewhere herein.

C. Administration Regimens According to certain embodiments of the present invention, multiple doses of a protein or peptide, e.g., recombinant relaxin-2, of the invention may be administered to a subject over a defined time course. The methods according to this aspect of the invention comprise sequentially administering to a subject multiple doses of a recombinant relaxin-2 of the invention. As used herein, “sequentially administering” means that each dose of a protein or peptide of the invention is administered to the subject at a different point in time, e.g., on different days separated by a predetermined interval (e.g., hours, days, weeks, or months). The present invention includes methods which comprise sequentially administering to the patient a single initial dose of a recombinant relaxin-2, followed by one or more secondary doses of the recombinant relaxin-2, and optionally followed by one or more tertiary doses of the recombinant relaxin-2.

The terms “initial dose,” “secondary doses,” and “tertiary doses,” refer to the temporal sequence of administration of the recombinant relaxin-2 of the invention. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the recombinant relaxin-2, but generally may differ from one another in terms of frequency of administration. In certain embodiments, however, the amount of a recombinant relaxin-2 contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of recombinant relaxin-2, which is administered to a patient prior to the administration of the very next dose in the sequence with no intervening doses.

The methods according to this aspect of the invention may comprise administering to a patient any number of secondary and/or tertiary doses of a protein or peptide (e.g., a recombinant relaxin-2). For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In one embodiment, the recombinant relaxin-2 is administered to a subject as a weight-based dose. A “weight-based dose” (e.g., a dose in mg/kg) is a dose of the protein or peptides that will change depending on the subject's weight.

In another embodiment, a protein or peptide, e.g., recombinant relaxin-2, is administered to a subject as a fixed dose. A “fixed dose” (e.g., a dose in mg) means that one dose of the protein or peptide, e.g., recombinant relaxin-2 is used for all subjects regardless of any specific subject-related factors, such as weight. In one particular embodiment, a fixed dose of a recombinant relaxin-2 of the invention is based on a predetermined weight or age.

In general, a suitable dose of the protein or peptide of the invention can be in the range of about 0.001 to about 200.0 milligram per kilogram body weight of the recipient, generally in the range of about 1 to 50 mg per kilogram body weight. For example, the protein or peptide, e.g., recombinant relaxin-2, can be administered at about 0.1 mg/kg, about 0.2 mg/kg, about 0.5 mg/kg, about 1 mg/kg, about 1.5 mg/kg, about 2 mg/kg, about 3 mg/kg, about 5 mg/kg, about 10 mg/kg, about 15 mg/kg, about 20 mg/kg, about 25 mg/kg, about 30 mg/kg, about 40 mg/kg, about 50 mg/kg per single dose. Values and ranges intermediate to the recited values are also intended to be part of this invention.

In some embodiments, the protein or peptide, e.g., the recombinant relaxin-2, of the invention is administered as a fixed dose of between about 10 mg to about 2500 mg. In some embodiments, the recombinant relaxin-2 of the invention is administered as a fixed dose of about 10 mg, about 15 mg, about 20 mg, 25 mg, about 30 mg, about 50 mg, about 75 mg, about 100 mg, about 125 mg, about 150 mg, about 175 mg, 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1500 mg, about 2000 mg, or about 2500 mg. Values and ranges intermediate to the recited values are also intended to be part of this invention.

### IV. Kits

Any of the compositions described herein may be comprised in a kit. In a non-limiting example, the kit comprises a recombinant relaxin-2.

The kit may further include reagents or instructions for using the recombinant relaxin-2 in a subject. It may also include one or more buffers.

The components of the kits may be packaged either in aqueous media or in lyophilized form. The container means of the kits will generally include at least one vial, test tube, flask, bottle, syringe, or other container means, into which a component may be placed, and preferably, suitably aliquoted. Where there is more than one component in the kit (labeling reagent and label may be packaged together), the kit also will generally contain a second, third or other additional container into which the additional components may be separately placed. The kits may also comprise a second container means for containing a sterile, pharmaceutically acceptable buffer and/or other diluent. However, various combinations of components may be comprised in a vial. The kits of the present invention also will typically include a means for containing the compositions of the invention, e.g., the recombinant relaxin-2, and any other reagent containers in close confinement for commercial sale.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The present invention is further illustrated by the following examples, which should not be construed as limiting. The entire contents of all of the references cited throughout this application are hereby expressly incorporated herein by reference.

### EXAMPLES

### Example 1. Recombinant Relaxin-2 Proteins

Engineered forms of relaxin-2 proteins that allow straightforward production in mammalian cells were designed. Briefly, single chain recombinant relaxin-2 proteins were designed to comprise, from N-terminus to C-terminus in operable order, relaxin B-linker-relaxin A. The recombinant relaxin-2 proteins optionally further comprise a second linker and/or a detectable label. The components, structures, and sequences of exemplary single chain recombinant relaxin-2 proteins are listed in Table 1 below. The single chain recombinant relaxin-2 proteins have several advantages, including, but not limited to, requiring no downstream processing or modification steps.

TABLE 1
Single Chain Recombinant Relaxin-2 Proteins

| | SEQ | | |
|---|---|---|---|
| Identi- | ID | Description | Sequences |
| fier | NO | | |
| SE001 | 25 | His-tag single-chain relaxin | HHHHHHDSWMEEVIKLCGRELVRAQIAICGMSTWSDAASSHSHS |
| | | | SARQLYSALANKCCHVGCTKRSLARFC |
| SE002 | 26 | His-tag single-chain relaxin | HHHHHHDSWMEEVIKLCGRELVRAQIAICGMSTWSDAASSHSHS |
| | | R41A Q42D¹ | SAADLYSALANKCCHVGCTKRSLARFC |
| SE003 | 27 | His-tag single-chain relaxin | HHHHHHGSLEVLFQGPGDSWMEEVIKLCGRELVRAQIAICGMST |
| | | with HRV 3C cleavage site | WSDAASSHSHSSARQLYSALANKCCHVGCTKRSLARFC |
| SE004 | 28 | Protein C-tag and His-tag | EDQVDPRLIDGKGSHHHHHHDSWMEEVIKLCGRELVRAQIAICG |
| | | single-chain relaxin | MSTWSDAASSHSHSSARQLYSALANKCCHVGCTKRSLARFC |
| SE005 | 29 | Truncated His-tag single-chain | HHHHHHEEVIKLCGRELVRAQIAICGMSTWSDAASSHSHSSARQL |
| | | relaxin | YSALANKCCHVGCTKRSLARFC |
| SE006 | 30 | Truncated His-tag single-chain | HHHHHHLEVLFQGPGEEVIKLCGRELVRAQIAICGMSTWSDAASS |
| | | relaxin with 3C cleavage site | HSHSSARQLYSALANKCCHVGCTKRSLARFC |
| SE101 | 31 | Single-chain relaxin fused to | DSWMEEVIKLCGRELVRAQIAICGMSTWSDAASSHSHSSARQLY |
| | | the N-terminus of human IgG1 Fc | SALANKCCHVGCTKRSLARFCGSGGSGDKTHTCPPCPAPELLGGP |
| | | | SVFLFPPKPKDTLMISRTPEVTCVVVDVSHEDPEVKFNWYVDGV |
| | | | EVHNAKTKPREEQYNSTYRVVSVLTVLHQDWLNGKEYKCKVSN |
| | | | KALPAPIEKTISKAKGQPREPQVYTLPPSREEMTKNQVSLTCLVK |
| | | | GFYPSDIAVEWESNGQPENNYKTTPPVLDSDGSFFLYSKLTVDKS |
| | | | RWQQGNVFSCSVMHEALHNHYTQKSLSLSPGK |
| SE102 | 32 | Single-chain relaxin fused to the | DKTHTCPPCPAPELLGGPSVFLFPPKPKDTLMISRTPEVTCVVVDV |
| | | C-terminus of human IgG1 Fc v.1 | SHEDPEVKFNWYVDGVEVHNAKTKPREEQYNSTYRVVSVLTVL |
| | | (short linker) | HQDWLNGKEYKCKVSNKALPAPIEKTISKAKGQPREPQVYTLPPS |
| | | | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPGKGGSDSWMEEVIKLCGRELVRAQIAICGMSTWSDAASSHS |
| | | | HSSARQLYSALANKCCHVGCTKRSLARFC |
| SE103 | 33 | Single-chain relaxin fused to the | DKTHTCPPCPAPELLGGPSVFLFPPKPKDTLMISRTPEVTCVVVDV |
| | | C-terminus of human IgG1 Fc v.2 | SHEDPEVKFNWYVDGVEVHNAKTKPREEQYNSTYRVVSVLTVL |
| | | (long linker) | HQDWLNGKEYKCKVSNKALPAPIEKTISKAKGQPREPQVYTLPPS |
| | | | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPGKGGSGGSGGSGGSDSWMEEVIKLCGRELVRAQIAICGMST |
| | | | WSDAASSHSHSSARQLYSALANKCCHVGCTKRSLARFC |
| SE201 | 34 | Single-chain relaxin fused to the | DKTHTCPPCPAPELLGGPSVFLFPPKPKDTLMISRTPEVTCVVVDV |
| | | C-terminus of human IgG1 Fc v.1 | SHEDPEVKFNWYVDGVEVHNAKTKPREEQYQSTYRVVSVLTVL |
| | | with N77Q Fc substitution² | HQDWLNGKEYKCKVSNKALPAPIEKTISKAKGQPREPQVYTLPPS |
| | | | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPGKGGSDSWMEEVIKLCGRELVRAQIAICGMSTWSDAASSHS |
| | | | HSSARQLYSALANKCCHVGCTKRSLARFC |
| SE202 | 35 | Single-chain relaxin fused to the | DKTHTCPPCPAPELLGGPSVFLFPPKPKDTLMISRTPEVTCVVVDV |
| | | C-terminus of human IgG1 Fc v.1 | SHEDPEVKFNWYVDGVEVHNAKTKPREEQYQSTYRVVSVLTVL |
| | | with N77Q Fc substitution and | HQDWLNGKEYKCKVSNKALPAPIEKTISKAKGQPREPQVYTLPPS |
| | | AAA linker between relaxin and | REEMTKNQVSLTCLVKGFYPSDIAVEWESNGQPENNYKTTPPVL |
| | | the Fc | DSDGSFFLYSKLTVDKSRWQQGNVFSCSVMHEALHNHYTQKSLS |
| | | | LSPGKAAADSWMEEVIKLCGRELVRAQIAICGMSTWSDAASSHS |
| | | | HSSARQLYSALANKCCHVGCTKRSLARFC |
| SE203 | 36 | Single-chain relaxin fused to the | DKTHTCPPCPAPELLGGPSVFLFPPKPKDTLMISRTPEVTCVVVDV |
| | | C-terminus of human IgG1 Fc v.1 | SHEDPEVKFNWYVDGVEVHNAKTKPREEQYQSTYRVVSVLTVL |
| | | with N77Q Fc substitution and | HQDWLNGKEYKCKVSNKALPAPIEKTISKAKGQPREPQVYTLPPS |
| | | PPP linker between relaxin and | REEMTKNQVSLTCLVKGFYPSDIAVEWESNGQPENNYKTTPPVL |
| | | the Fc | DSDGSFFLYSKLTVDKSRWQQGNVFSCSVMHEALHNHYTQKSLS |
| | | | LSPGKPPPDSWMEEVIKLCGRELVRAQIAICGMSTWSDAASSHSH |
| | | | SSARQLYSALANKCCHVGCTKRSLARFC |
| SE204 | 37 | Single-chain relaxin fused to the | DKTHTCPPCPAPELLGGPSVFLFPPKPKDTLMISRTPEVTCVVVDV |
| | | C-terminus of human IgG1 Fc v.1 | SHEDPEVKFNWYVDGVEVHNAKTKPREEQYQSTYRVVSVLTVL |
| | | with N77Q Fc substitution and | HQDWLNGKEYKCKVSNKALPAPIEKTISKAKGQPREPQVYTLPPS |
| | | M4K relaxin substitution | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPGKGGSDSWKEEVIKLCGRELVRAQIAICGMSTWSDAASSHSH |
| | | | SSARQLYSALANKCCHVGCTKRSLARFC |
| SE205 | 38 | Single-chain relaxin fused to the | DKTHTCPPCPAPELLGGPSVFLFPPKPKDTLMISRTPEVTCVVVDV |
| | | C-terminus of human IgG1 Fc v.1 | SHEDPEVKFNWYVDGVEVHNAKTKPREEQYQSTYRVVSVLTVL |
| | | with N77Q Fc substitution and | HQDWLNGKEYKCKVSNKALPAPIEKTISKAKGQPREPQVYTLPPS |
| | | M25K relaxin substitution | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPGKGGSDSWMEEVIKLCGRELVRAQIAICGKSTWSDAASSHSH |
| | | | SSARQLYSALANKCCHVGCTKRSLARFC |
| SE206 | 39 | Single-chain relaxin fused to the | DKTHTCPPCPAPELLGGPSVFLFPPKPKDTLMISRTPEVTCVVVDV |
| | | C-terminus of human IgG1 Fc v.1 | SHEDPEVKFNWYVDGVEVHNAKTKPREEQYQSTYRVVSVLTVL |
| | | with N77Q Fc substitution and | HQDWLNGKEYKCKVSNKALPAPIEKTISKAKGQPREPQVYTLPPS |
| | | W28A relaxin substitution | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPGKGGSDSWMEEVIKLCGRELVRAQIAICGMSTASDAASSHSH |
| | | | SSARQLYSALANKCCHVGCTKRSLARFC |
| SE207 | 40 | Single-chain relaxin fused to the | DKTHTCPPCPAPELLGGPSVFLFPPKPKDTLMISRTPEVTCVVVDV |
| | | C-terminus of human IgG1 Fc v.1 | SHEDPEVKFNWYVDGVEVHNAKTKPREEQYQSTYRVVSVLTVL |
| | | with N77Q Fc substitution and | HQDWLNGKEYKCKVSNKALPAPIEKTISKAKGQPREPQVYTLPPS |
| | | redesigned single-chain relaxin | REEMTKNQVSLTCLVKGFYPSDIAVEWESNGQPENNYKTTPPVL |
| | | linker | DSDGSFFLYSKLTVDKSRWQQGNVFSCSVMHEALHNHYTQKSLS |
| | | | LSPGKGGSDSWMEEVIKLCGRELVRAQIAICGMSTWSDAAGANA |
| | | | NAGARQLYSALANKCCHVGCTKRSLARFC |
| SE301 | 41 | Single-chain relaxin fused to the | DKTHTCPPCPAPELLGGPSVFLFPPKPKDTLMISRTPEVTCVVVDV |
| | | C-terminus of human IgG1 Fc | SHEDPEVKFNWYVDGVEVHNAKTKPREEQYQSTYRVVSVLTVL |
| | | with combined substitutions of | HQDWLNGKEYKCKVSNKALPAPIEKTISKAKGQPREPQVYTLPPS |
| | | SE201, SE204-207 | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPGKGGSDSWKEEVIKLCGRELVRAQIAICGKSTASDAAGANA |
| | | | NAGARQLYSALANKCCHVGCTKRSLARFC |
| SE302 | 42 | Single-chain relaxin fused to the | DKTHTCPPCPAPELLGGPSVFLFPPKPKDTLMISRTPEVTCVVVDV |
| | | C-terminus of human IgG1 Fc | SHEDPEVKFNWYVDGVEVHNAKTKPREEQYQSTYRVVSVLTVL |
| | | with combined substitutions of | HQDWLNGKEYKCKVSNKALPAPIEKTISKAKGQPREPQVYTLPPS |
| | | SE201, 204-207 and no linker | REEMTKNQVSLTCLVKGFYPSDIAVEWESNGQPENNYKTTPPVL |
| | | between the Fc and single-chain | DSDGSFFLYSKLTVDKSRWQQGNVFSCSVMHEALHNHYTQKSLS |
| | | relaxin | LSPGKDSWKEEVIKLCGRELVRAQIAICGKSTASDAAGANANAG |
| | | | ARQLYSALANKCCHVGCTKRSLARFC |
| SE303 | 43 | Single-chain relaxin fused to the | DKTHTCPPCPAPELLGGPSVFLFPPKPKDTLMISRTPEVTCVVVDV |
| | | C-terminus of human IgG1 Fc | SHEDPEVKFNWYVDGVEVHNAKTKPREEQYQSTYRVVSVLTVL |
| | | with combined substitutions of | HQDWLNGKEYKCKVSNKALPAPIEKTISKAKGQPREPQVYTLPPS |
| | | SE201, 204-207 and R13D R17D | REEMTKNQVSLTCLVKGFYPSDIAVEWESNGQPENNYKTTPPVL |
| | | relaxin substitutions to reduce | DSDGSFFLYSKLTVDKSRWQQGNVFSCSVMHEALHNHYTQKSLS |
| | | activity | LSPGKGGSDSWKEEVIKLCGDELVDAQIAICGKSTASDAAGANA |
| | | | NAGARQLYSALANKCCHVGCTKRSLARFC |
| SE304 | 44 | Single-chain relaxin fused to the | DKTHTCPPCPAPELLGGPSVFLFPPKPKDTLMISRTPEVTCVVVDV |
| | | C-terminus of human IgG1 Fc | SHEDPEVKFNWYVDGVEVHNAKTKPREEQYQSTYRVVSVLTVL |
| | | with combined substitutions of | HQDWLNGKEYKCKVSNKALPAPIEKTISKAKGQPREPQVYTLPPS |
| | | SE201, SE204-207 and R13D | REEMTKNQVSLTCLVKGFYPSDIAVEWESNGQPENNYKTTPPVL |
| | | R17D I20D relaxin substitutions | DSDGSFFLYSKLTVDKSRWQQGNVFSCSVMHEALHNHYTQKSLS |
| | | to reduce activity | LSPGKGGSDSWKEEVIKLCGDELVDAQDAICGKSTASDAAGANA |
| | | | NAGARQLYSALANKCCHVGCTKRSLARFC |
| SE305 | 45 | Single-chain relaxin fused to the | DKTHTCPPCPAPELLGGPSVFLFPPKPKDTLMISRTPEVTCVVVDV |
| | | C-terminus of human IgG1 Fc | SHEDPEVKFNWYVDGVEVHNAKTKPREEQYQSTYRVVSVLTVL |
| | | with combined substitutions of | HQDWLNGKEYKCKVSNKALPAPIEKTISKAKGQPREPQVYTLPPS |
| | | SE201, SE204-207 and R13A | REEMTKNQVSLTCLVKGFYPSDIAVEWESNGQPENNYKTTPPVL |
| | | R17A I20A relaxin substitutions | DSDGSFFLYSKLTVDKSRWQQGNVFSCSVMHEALHNHYTQKSLS |
| | | to reduce activity | LSPGKGGSDSWKEEVIKLCGAELVAAQAAICGKSTASDAAGANA |
| | | | NAGARQLYSALANKCCHVGCTKRSLARFC |
| SE401 | 46 | Single-chain relaxin with the | HHHHHHHHRGVFRREAHKSEIAHRYNDLGEQHFKGLVLIAFSQY |
| | | combined substitutions of SE201, | LQKCSYDEHAKLVQEVTDFAKTCVADESAANCDKSLHTLFGDK |
| | | 204-207, fused to the C-terminus | LCAIPNLRENYGELADCCTKQEPERNECFLQHKDDNPSLPPFERPE |
| | | of mouse serum albumin with an | AEAMCTSFKENPTTFMGHYLHEVARRHPYFYAPELLYYAEQYNE |
| | | N-terminal 8X His-tag (SEQ ID | ILTQCCAEADKESCLTPKLDGVKEKALVSSVRQRMKCSSMQKFG |
| | | NO: 19) | ERAFKAWAVARLSQTFPNADFAEITKLATDLTKVNKECCHGDLL |
| | | | ECADDRAELAKYMCENQATISSKLQTCCDKPLLKKAHCLSEVEH |
| | | | DTMPADLPAIAADFVEDQEVCKNYAEAKDVFLGTFLYEYSRRHP |
| | | | DYSVSLLLRLAKKYEATLEKCCAEANPPACYGTVLAEFQPLVEEP |
| | | | KNLVKTNCDLYEKLGEYGFQNAILVRYTQKAPQVSTPTLVEAAR |
| | | | NLGRVGTKCCTLPEDQRLPCVEDYLSAILNRVCLLHEKTPVSEHV |
| | | | TKCCSGSLVERRPCFSALTVDETYVPKEFKAETFTFHSDICTLPEK |
| | | | EKQIKKQTALAELVKHKPKATAEQLKTVMDDFAQFLDTCCKAA |
| | | | DKDTCFSTEGPNLVTRCKDALAGGSDSWKEEVIKLCGRELVRAQ |
| | | | IAICGKSTASDAAGANANAGARQLYSALANKCCHVGCTKRSLA |
| | | | RFC |
| SE501 | 60 | Single-chain relaxin fused to the | DKTHTCPPCPAPELLGGPSVFLFPPKPKDTLMISRTPEVTCVVVDV |
| | | C-terminus of human IgG1 Fc | SHEDPEVKFNWYVDGVEVHNAKTKPREEQYQSTYRVVSVLTVL |
| | | with combined substitutions of | HQDWLNGKEYKCKVSNKALPAPIEKTISKAKGQPREPQVYTLPPS |
| | | SE301 and A264S | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPGKGGSDSWKEEVIKLCGRELVRSQIAICGKSTASDAAGANA |
| | | | NAGARQLYSALANKCCHVGCTKRSLARFC |
| SE502 | 61 | Single-chain relaxin fused to the | DKTHTCPPCPAPELLGGPSVFLFPPKPKDTLMISRTPEVTCVVVDV |
| | | C-terminus of human IgG1 Fc | SHEDPEVKFNWYVDGVEVHNAKTKPREEQYQSTYRVVSVLTVL |
| | | with combined substitutions of | HQDWLNGKEYKCKVSNKALPAPIEKTISKAKGQPREPQVYTLPPS |
| | | SE301 and A264G | REEMTKNQVSLTCLVKGFYPSDIAVEWESNGQPENNYKTTPPVL |
| | | | DSDGSFFLYSKLTVDKSRWQQGNVFSCSVMHEALHNHYTQKSLS |
| | | | LSPGKGGSDSWKEEVIKLCGRELVRGQIAICGKSTASDAAGANA |
| | | | NAGARQLYSALANKCCHVGCTKRSLARFC |

¹The representation is similar to the representation for the description of substitutions in relaxin A and relaxin B, except that the counting of the amino acid location starts at the first amino acid of relaxin B in the single chain relaxin-2.
²For Fc fragment, the representation is similar to the representation for the description of substitutions in relaxin A and relaxin B, except that the counting of the amino acid location starts at the first amino acid of Fc fragment independent of the relaxin B-linker-relaxin A peptide.

### Example 2. Generation of Recombinant Single Chain Relaxin-2 Proteins

Standard molecular biology techniques were used to generate recombinant relaxin-2 proteins of the invention. Briefly, DNA encoding any one of the proteins listed in Table 1, were operably linked and cloned into an inducible pcDNA3.1-Zeo-tetO expression plasmid or pFUSE-hIgG1-Fc2 plasmid. The recombinant plasmid was transfected using ExpiFectamine or polyethylenimine (PEI) into Expi293F cells. For some recombinant plasmids, a tetracycline-inducible stable cell line was generated with Expi293F cells for expression of the recombinant relaxin-2 proteins. Cell culture and transfection were performed according to the manufacturer's manual. Cells were harvested and the total proteins were collected using techniques well known in the art. The recombinant relaxin-2 proteins were purified using affinity chromatography or affinity chromatography followed by size exclusion chromatography. Immobilized metal affinity chromatography (IMAC) and size exclusion chromatography were used for recombinant relaxin-2 proteins containing a 6×His tag (SEQ ID NO: 18) or 8×His tag (SEQ ID NO: 19). Protein G antibody affinity chromatography was used for recombinant relaxin-2 proteins containing an IgG1 Fc tag.

### Example 3. Biochemical Data for Single Chain Relaxin-2 Proteins

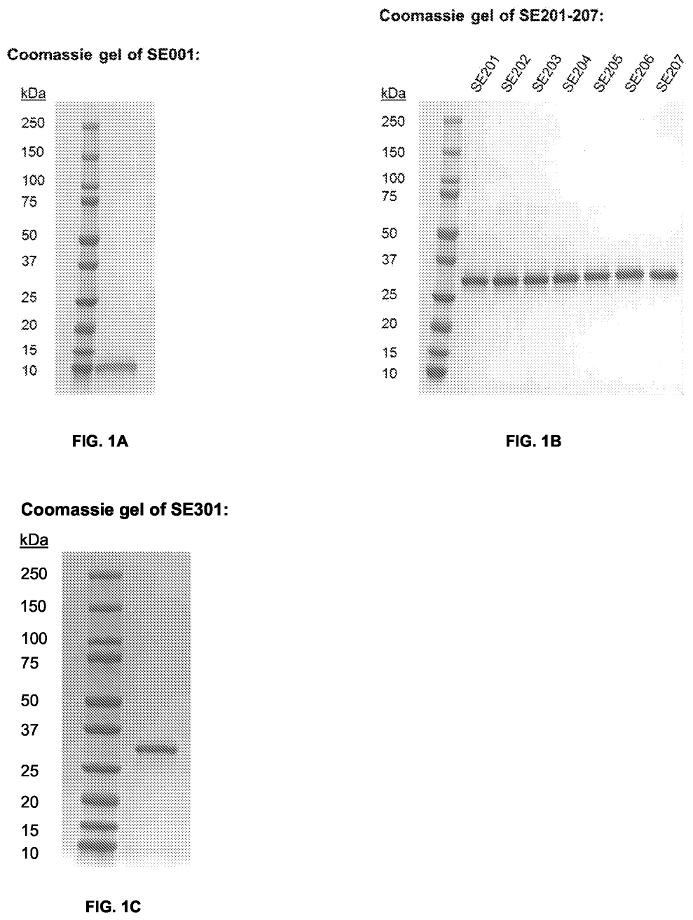

Purified recombinant single chain relaxin-2 proteins were subject to SDS-PAGE electrophoresis and Coomassie blue staining to determine the molecular weights and purity. As shown in FIGS. 1A-1C, exemplary recombinant single chain protein SE001 has the expected molecular weight of about 8 kDa; and exemplary recombinant single chain proteins SE201, SE202, SE203, SE204, SE205, SE206, SE207, and SE301 have the expected molecular weight of about 32 kDa. The Coomassie blue staining also demonstrated that the purified recombinant single chain relaxin-2 proteins were substantially free of contaminant proteins.

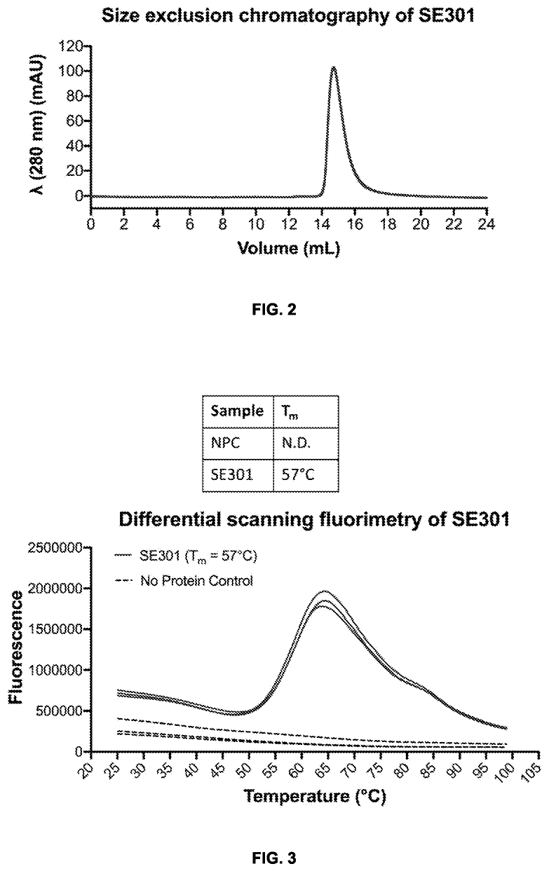

For SE301, size exclusion chromatography of the protein following affinity chromatography was monitored by measuring the absorption at 280 nm of the eluted fractions. FIG. 2 demonstrated that SE301 purified by affinity chromatography was substantially free of contaminant proteins.

The melting temperature (Tₘ) of SE301 was determined using differential scanning fluorimetry. As shown in FIG. 3, the Tₘ of SE301 is about 57° C.

### Example 4. Activities of Recombinant Relaxin-2 Proteins

The biological activities of the recombinant relaxin-2 proteins were tested using cAMP driven report gene assay. In the reporter gene assay, the recombinant relaxin-2 proteins bind to the RXFP1 receptor that has been expressed by transient transfection in HEK293T cells. Binding of the recombinant relaxin-2 proteins activates RXFP1, causing an increase in cAMP levels in the cells. The cAMP signaling cascade leads to the activation of a promoter with cAMP response elements (CRE). The promoter controls transcription of the reporter gene for the enzyme secreted embryonic alkaline phosphatase (SEAP). As a result, SEAP is produced and secreted into the cell culture medium by the HEK293T cells. The substrate for SEAP, 4-methylumbelliferyl phosphate (MUP), is then mixed with the medium. According to the amount of SEAP present in the medium, the reaction can lead to the enzymatic creation of a fluorescent product which is detected by a plate reader. Therefore, the fluorescence reading used to detect the level of SEAP enzyme acts as a readout for the recombinant relaxin-2 induced activation of RXFP1 in a cell. Cell culture and transfection were conducted according to the manufacturer's manual. The cAMP driven report gene assay was described in Durocher et al., A Report Gene Assay for High-Throughput Screen of G-protein-coupled Receptors Stably or Transiently Expressed in HEK293 EBNA Cells Grown in Suspension Culture, Anal. Biochem., 284(2):316-26 (2000), and in Liberles & Buck, A Second Class of Chemosensory Receptors in the Olfactory Epithelium, Nature, 442(7103): 645-50 (2006), incorporated herein by reference.

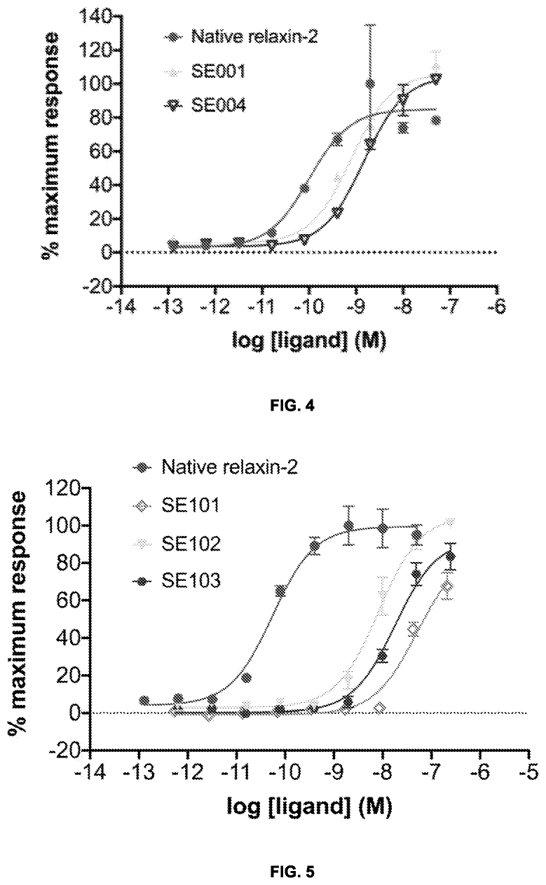

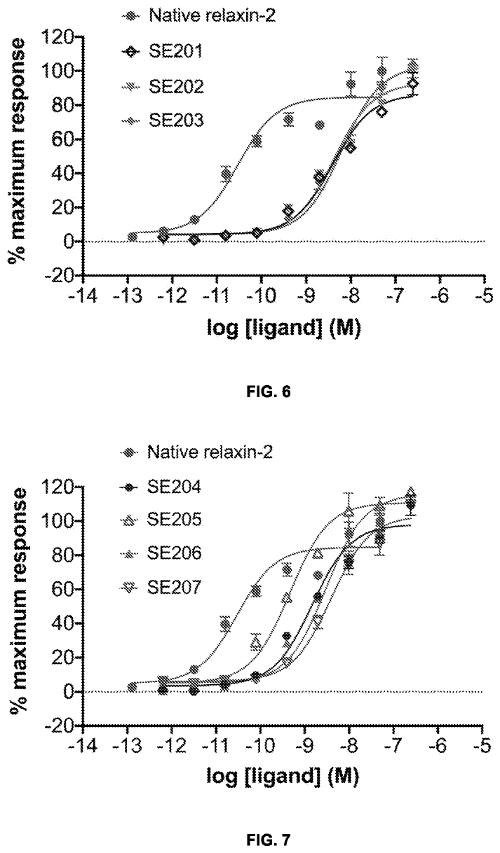

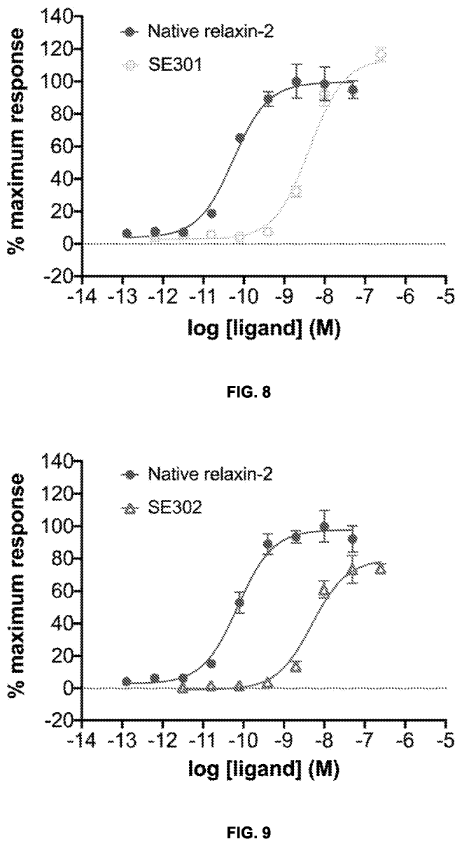

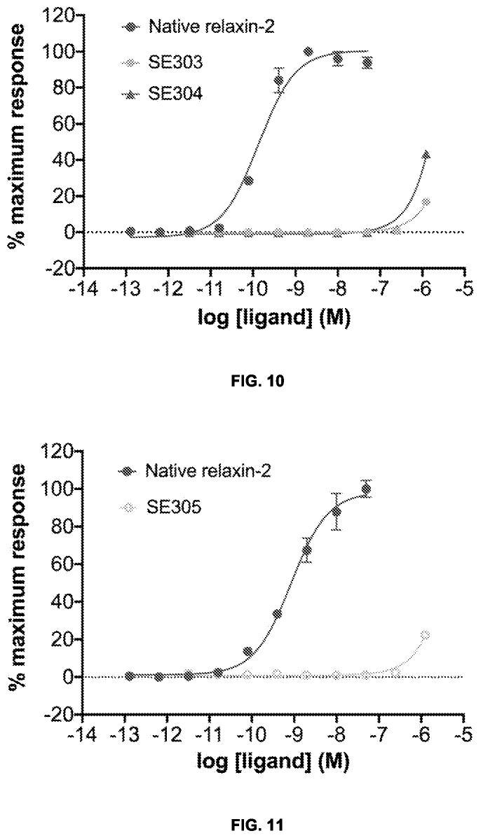

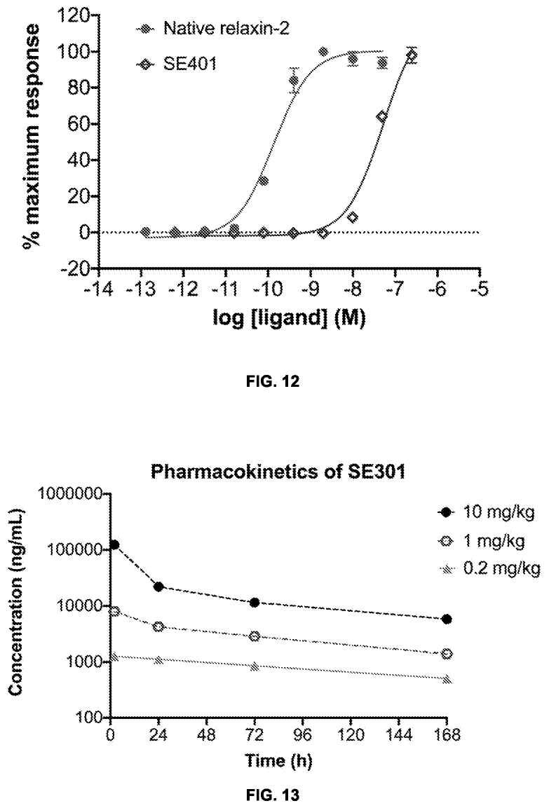

The activities and EC₅₀s of the single chain recombinant relaxin-2 were summarized in Table 2 and in FIGS. 4-12.

TABLE 2
Activities of Recombinant Relaxin-2 Proteins

| | | Emax | | |
|---|---|---|---|---|
| | | (Native | | |
| | | two- | | Bio- |
| | | chain | | chemical |
| | | relaxin = | | Yield |
| | | 100% | Expression | (mg/L |
| Identifier | EC₅₀ | Emax) | Plasmid | culture) |
| SE001 | 85 pM- | 118% | pcDNA3.1* | 0.5 |
| | 793 pM | | | |
| SE002 | N.D. | N.D. | pcDNA3.1* | |
| SE003 | N.D. | N.D. | pcDNA3.1* | 0.4 |
| SE004 | 1.5 nM | 117% | pcDNA3.1* | 2.1 |
| SE005 | 485 pM | 112% | pcDNA3.1* | 0.4 |
| SE006 | N.D. | N.D. | pcDNA3.1* | 0.4 |
| SE101 | 49.4 nM | 86% | pFUSE-hIgG1-Fc2 | 14 |
| SE102 | 8.3 nM | 106% | pcDNA3.1* | 178 |
| SE103 | 17.9 nM | 93% | pcDNA3.1* | 216 |
| SE201 | 3.9 nM | 102% | pcDNA3.1* | |
| SE202 | 4.2 nM | 109% | pcDNA3.1* | |
| SE203 | 6.3 nM | 121% | pcDNA3.1* | |
| SE204 | 1.5 nM | 115% | pcDNA3.1* | |
| SE205 | 465 nM | 130% | pcDNA3.1* | |
| SE206 | 3.0 nM | 135% | pcDNA3.1* | |
| SE207 | 3.9 nM | 121% | pcDNA3.1* | |
| SE301 | 4.2 nM | 114% | pcDNA3.1* | 156 |
| SE302 | 4.9 nM | 81% | pcDNA3.1* | 125 |
| SE303 | N.A. | N.A. | pcDNA3.1* | 43 |
| SE304 | N.A. | N.A. | pcDNA3.1* | 104 |
| SE305 | N.A. | N.A. | pcDNA3.1* | 200 |
| SE401 | 52.6 nM | 121% | pcDNA3.1* | 82 |

*modified pcDNA3.1 plasmid: pcDNA3.1-Zeo-tetO inducible expression plasmid.

### Example 5. Pharmacokinetics Study of Recombinant Relaxin-2 Proteins

To determine the serum pharmacokinetics of SE301, a pharmacokinetics study following single intraperitoneal injection administration to male CD-1 mice was conducted. Stock formulation of purified SE301 was prepared at 10 mg/mL in sterile phosphate buffered saline and stored at −80° C.

On the day before dosing day, the stock formulation of SE301 was diluted according to Table 3 below. The diluted formulation injection was dispensed under a laminar flow hood for dosing if needed. Dose formulation analysis was conducted the day before dosing using a non validated method. The stability (24 hours at room temperature) of the test article (the SE301 formulation) was established before the start of study. The test article was allowed to warm to room temperature at least 30 minutes before dosing but not longer than 3 hours when not in use.

Nine male CD-1 mice were used in the study. Each mouse was between about 7 to about 10 weeks of age at the dosing day and weighed between about 29 and about 40 grams. Animal husbandry and clinical observation were conducted according to established protocol at the test facility. The experimental design was shown in Table 3 below.

TABLE 3

| | No. of | Treatment | | | | | |
|---|---|---|---|---|---|---|---|
| | Mice | | | | Dose | | |
| | (#/gender) | Test | Dose | Conc. | Volume | | Dose |
| Group # | Male | Article | (mg/kg) | (mg/mL) | (mL/kg) | Vehicle | Route |
| 1 | 3 | SE301 | 1 | 0.2 | 5 | PBS | Single IP |
| 2 | 3 | SE301 | 5 | 1 | 5 | PBS | Single IP |
| 3 | 3 | SE301 | 10 | 10 | 5 | PBS | Single IP |

Note:
1. The first dosing day will be assigned as Day 1.

The intraperitoneal injection (IP) dose was administered via hypogastric regions. Animals were weighed prior to dose administration and dose volume was adjusted based on the body weights. The blood samples from the test mice were collected at pre-dose, 2 hours, 24 hours, 72 hours, and 168 hours post-dose.

For control serum, blood samples from male animals were collected from inferior vena cava. Whole blood was collected from available CD-1 mice into commercially available tubes containing polymer silica activator. The vacutainer tubes containing blood samples remained at room temperature for 30 minutes before centrifugation (after serum appeared). The samples were centrifuged at 4° C. for 15 minutes at 2,500×g within one hour of collection. The sera were transferred into a pre-labeled polyethylene micro centrifuge tube. About 5 mL total male serum was collected. The serum was stored at −60° C. or lower immediately until bio-analysis or shipment. The serum served as control serum for bio-analysis.

To prepare serum samples for PK analysis, at least 0.6 mL blood sample was collected at sampling time points from each animal in test compound treatment groups. For samples collected within the first hour of dosing, ±1 minute was deemed acceptable. For the remaining time points, samples that were taken within 5% of the scheduled time were deemed acceptable and were not considered as protocol deviation. All blood samples were collected into commercially available tubes containing polymer silica activator. After blood was collected, the tubes containing blood samples remained at room temperature for around 30 minutes before centrifugation (after serum appeared). The samples were centrifuged at 4° C. for 15 minutes at 2,500×g within one hour of collection. Then serum was collected after centrifugation, and one aliquot (at least 30 μL) was made for PK analysis. The samples were then quickly frozen over dry ice and kept at −60° C. or lower until analysis. A qualified ELISA was conducted to analyze the amount of SE301.

Serum concentration versus time data and derived pharmacokinetic parameters were analyzed by non-compartmental approaches using the WinNonlin software program.

The result of the pharmacokinetics study was shown in FIG. 13 and Table 4 below. As shown in FIG. 13 and Table 4, the circulating half-life of SE301 is about 77.5 hours at 10 mg/kg dosing, about 90.7 hours at 1 mg/kg dosing, and about 130 hours at 0.2 mg/kg dosing.

TABLE 4

| Dose | T_{1/2} |
|---|---|
| 0.2 mg/kg | 130 hours |
| 1 mg/kg | 90.7 hours |
| 10 mg/kg | 77.5 hours |

### Example 6. Activities of Recombinant Relaxin-2 Proteins

According to the method as described in Example 4, the biological activities of two recombinant relaxin-2 proteins, SE501 and SE502, were tested using cAMP driven report gene assay.

The amino acid sequence of the single chain recombinant relaxin-2 were summarized in Table 1.

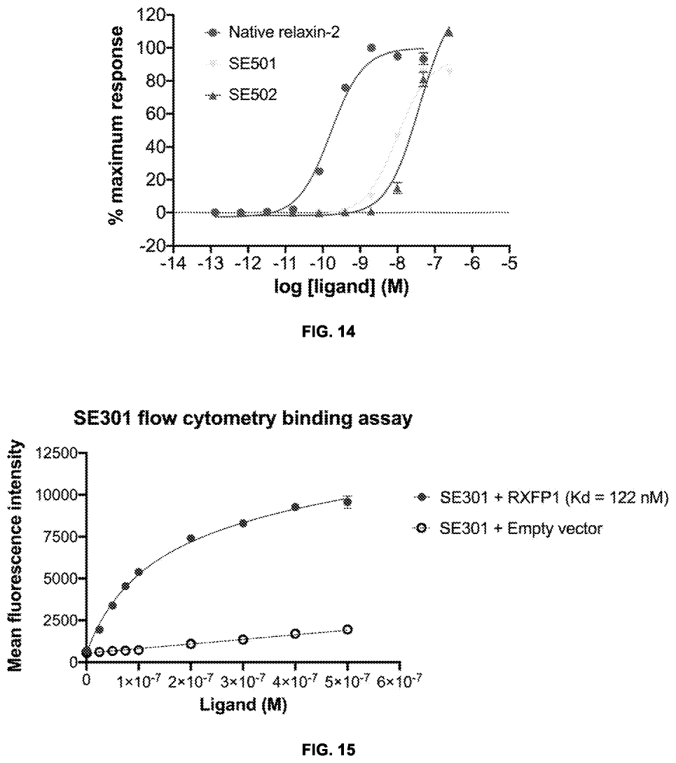

The activities and EC₅₀s of the single chain recombinant relaxin-2 were summarized in Table 5 and in FIG. 14.

TABLE 5
Activities of Recombinant Relaxin-2 Proteins

| | | Emax | | Bio- |
|---|---|---|---|---|
| | | (Native two- | | chemical |
| | | chain | | Yield |
| | | relaxin = | Expression | (mg/L |
| Identifier | EC₅₀ | 100% Emax) | Plasmid | culture) |
| SE501 | 9.7 nM | 94% | pcDNA3.1* | 50 |
| SE502 | 38.6 nM | 131% | pcDNA3.1* | 29 |

*modified pcDNA3.1 plasmid: pcDNA3.1-Zeo-tetO inducible expression plasmid.

### Example 7. Flow Cytometry Binding Assay for Recombinant Relaxin-2 Proteins

The binding affinity of SE301 was determined by a flow cytometry assay with Expi293F cells transiently transfected with either RXFP1 with an N-terminal FLAG tag or empty vector plasmids. Cell culture and transfection were conducted according to the manufacturer's manual. Cells transfected with RXFP1 or the empty vector control were incubated in a buffer of 20 mM HEPES pH 7.5, 150 mM sodium chloride, 2 mM calcium chloride, and 1% fetal bovine serum for 30 minutes at 4° C. Different concentrations of SE301 were added to the cells and incubated for 1 hour at 4° C. Cells were washed twice with buffer and an M1 antibody labeled with Alexa 488 (M1-488) and a secondary anti-human Fc antibody labeled with Alexa 647 (anti-human Fc-647) were incubated with the cells for 30 minutes at 4° C. The cells were washed once, resuspended in 100 μL buffer, and analyzed by flow cytometry. The cells were gated by forward scatter area versus side scatter area and forward scatter area versus forward scatter height. The cells were then gated according to receptor expression indicated by binding of the M1-488 antibody to the receptor's FLAG tag. Cells in the final gate were plotted according to mean fluorescence intensity of the anti-human Fc-647 antibody to calculate the Kd of SE301.

The results of the flow cytometry study was shown in FIG. 15. As shown in FIG. 15, the binding affinity (Kd) of SE301 for RXFP1 is 122 nM.

### INCORPORATION BY REFERENCE

All publications, patents, and patent applications mentioned herein are hereby incorporated by reference in their entirety as if each individual publication, patent, or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the present invention described herein. Such equivalents are intended to be encompassed by the following claims.

1. A fusion protein comprising, from N-terminus to C-terminus,
a first peptide comprising an amino acid sequence that is at least about 85% identical to the entire amino acid sequence of SEQ ID NO: 2;
a peptide linker comprising an amino acid sequence that is at least about 85% identical to the entire amino acid sequence of an amino acid sequence selected from the group consisting of DAASSHSHSSAR (SEQ ID NO: 14) and DAAGANANAGAR (SEQ ID NO: 16); and
a second peptide comprising an amino acid sequence that is at least about 85% identical to the entire amino acid sequence of SEQ ID NO: 1;
wherein the first peptide, the peptide linker, and the second peptide are operably linked.

2.-6. (canceled)

7. The fusion protein of claim 1, wherein the peptide linker comprises an amino acid sequence that is at least about 90% identical to the entire amino acid sequence of an amino acid sequence selected from the group consisting of DAASSHSHSSAR (SEQ ID NO: 14), DAASSHSHSSAA (SEQ ID NO: 15), and DAAGANANAGAR (SEQ ID NO: 16).

8. (canceled)

9. The fusion protein of claim 1, wherein the first peptide has an amino acid sequence that is at least about 95% identical to the entire amino acid sequence of SEQ ID NO: 2, and the second peptide has an amino acid sequence that is at least about 95% identical to the entire amino acid sequence of SEQ ID NO: 1, wherein the fusion protein has a native relaxin-2 activity.

10.-12. (canceled)

13. A fusion protein comprising a first peptide, a peptide linker, and a second peptide, wherein the amino acid sequence of the fusion protein is at least about 85% identical to the entire amino acid sequence of an amino acid sequence selected from the group consisting of SEQ ID NOs: 47, 48, 49, 50, 51, 52, 53, 54, and 55.

14. (canceled)

15. (canceled)

16. The fusion protein of claim 1, further comprising a first detectable label.

17.-30. (canceled)

31. The fusion protein of claim 1, further comprising a cleavable linker.

32.-34. (canceled)

35. The fusion protein of claim 1, further comprising a signal peptide at the N-terminus of the fusion protein.

36.-38. (canceled)

39. A peptide linker comprising an amino acid sequence having at least about 85% amino acid identity to the entire amino acid sequence of an amino acid selected from the group consisting of DAASSHSHSSAR (SEQ ID NO: 14) and DAAGANANAGAR (SEQ ID NO: 16).

40.-45. (canceled)

46. A polypeptide comprising an amino acid sequence that is at least about 90% identical to the entire amino acid sequence of SEQ ID NO:10.

47.-55. (canceled)

56. A polynucleotide comprising a nucleotide sequence encoding the fusion protein of claim 1, or the polypeptide of claim 46.

57. (canceled)

58. An expression vector comprising the polynucleotide of claim 56.

59. (canceled)

60. (canceled)

61. A recombinant cell comprising the polynucleotide of claim 56 or the expression vector of claim 58.

62.-67. (canceled)

68. A pharmaceutical composition comprising an effective amount of the fusion protein of claim 45, or the polypeptide of claim 46, or the polynucleotide of claim 56, or the expression vector of claim 58.

69. A method of enhancing a relaxin-2-related activity in a cell, comprising contacting the cell with the fusion protein of claim 45, or the polypeptide of claim 46, thereby enhancing relaxin-2-related activity in the cell.

70.-76. (canceled)

77. A method of treating a relaxin-associated disorder in a subject in need thereof, comprising administering to the subject an effective amount of the fusion protein of claim 45, or the polypeptide of claim 46, or the polynucleotide of claim 56, or the expression vector of claim 58, or the pharmaceutical composition of claim 68, thereby treating the relaxin-associated disorder.

78.-80. (canceled)

81. A kit comprising an effective amount of the fusion protein of claim 45, or the polypeptide of claim 46, or the polynucleotide of claim 56, or the expression vector of claim 58, or the pharmaceutical composition of claim 68, and an instruction of use.
